SEQUENCE LISTING (10 sequences)

SEQ ID NO: 1 - protein, 120 aa, synthetic construct
  REGION 1..120: note = Synthetic heavy chain variable domain
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 2 - protein, 111 aa, synthetic construct
  REGION 1..111: note = Synthetic light chain variable domain
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLG

SEQ ID NO: 3 - protein, 251 aa, synthetic construct
  REGION 1..251: note = Synthetic single-chain antibody
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGSGGGGSGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 4 - protein, 252 aa, synthetic construct
  REGION 1..252: note = Synthetic single-chain antibody
MEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGSGGGGSGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 5 - protein, 11 aa, synthetic construct
  REGION 1..11: note = CDRL1
QASEIIHSWLA

SEQ ID NO: 6 - protein, 7 aa, synthetic construct
  REGION 1..7: note = CDRL2
LASTLAS

SEQ ID NO: 7 - protein, 12 aa, synthetic construct
  REGION 1..12: note = CDRL3
QNVYLASTNGAN

SEQ ID NO: 8 - protein, 11 aa, synthetic construct
  REGION 1..11: note = CDRH1
GFSLTDYYYMT

SEQ ID NO: 9 - protein, 16 aa, synthetic construct
  REGION 1..16: note = CDRH2
FIDPDDDPYYATWAKG

SEQ ID NO: 10 - protein, 11 aa, synthetic construct
  REGION 1..11: note = CDRH3
GDHNSGWGLDI

(19) United States
(12) Patent Application Publication
(10) Pub. No.: US 2025/0197486 A1
(43) Pub. Date: Jun. 19, 2025

(54) METHODS FOR TREATING NEOVASCULAR AGE-RELATED MACULAR DEGENERATION

(71) Applicant: Novartis AG, Basel (CH)

(72) Inventors: Jeffrey David KEARNS, Arlington, MA (US); Zufar MULYUKOV, Basel (CH); Ufuk OLGAC, Zurich (CH); Etienne PIGEOLET, Rantzwiller (FR)

(21) Appl. No.: 18/846,943

(22) PCT Filed: Mar. 16, 2023
(86) PCT No.: PCT/IB2023/052565

§ 371 Date: Sep. 13, 2024

Related U.S. Application Data
(60) Provisional application No. 63/320,796, filed on Mar. 17, 2022.
(60) Provisional application No. 63/386,380, filed on Dec. 7, 2022.

(51) Int. Cl. C07K 16/22

(52) U.S. Cl. CPC C07K 16/22; C07K 2317/569; C07K 2317/92

(57) ABSTRACT

The invention relates to methods for treating neovascular age-related macular degeneration (nAMD) in a patient, the methods comprising (a) administering to the patient as a loading phase of two individual doses of a VEGF antagonist at 6-week interval (q6w regimen), (b) assessing the patient for disease activity after the second dose of the loading phase, and (c) optionally if presence of disease activity is identified after the second dose of the VEGF antagonist, the method further comprises administering to the patient as part of the loading phase a third dose of the VEGF antagonist 6 weeks after the second dose.

### FIELD

The invention relates to methods for treating neovascular age-related macular degeneration (nAMD) in a patient.

### BACKGROUND

Age-related macular degeneration (AMD) is the leading cause of severe vision loss in people affecting 10%-13% of individuals over the age of 65 in North America, Europe, and Australia (Kawasaki 2010, Rein et al., Arch Ophthalmol. 2009;127:533-40, Smith 2001). Genetic, environmental and health factors play an important role in the pathogenesis of the disease.

AMD is classified into 2 clinical subtypes: the non-neovascular (atrophic) or dry form and the neovascular (exudative) or wet form (Ferris et al., Arch Ophthalmol. 1984; 102:1640-2, Lim et al., Lancet. 2012;379:1728-38, Miller et al., Am J Ophthalmol. 2013;155:1-35). Neovascular AMD (nAMD) is characterized by the growth of abnormal new blood vessels (neovascularization) under the retinal pigment epithelium (RPE) or subretinal space from the subjacent choroid, termed choroidal neovascularization (CNV) (Ferris et al., Arch Ophthalmol. 1984;102:1640-2). These newly formed vessels have an increased likelihood to leak blood and serum, damaging the retina by stimulating inflammation and scar tissue formation. This damage to the retina results in progressive, severe, and irreversible vision loss (Shah et al., Am J Ophthalmol. 2007;143:83-89, Shah et al., Am J Ophthalmol. 2009;116:1901-07). Without treatment, most affected eyes will have poor central vision (20/200) within 12 months (TAP 2003). Although the neovascular form of the disease is only present in about 10% of all AMD cases, it accounted for approximately 90% of the severe vision loss from AMD prior to the introduction of anti-vascular endothelial growth factor (VEGF) treatments (Ferris et al., Am J Ophthalmol. 1983;118:132-51, Sommer et al., N Engl J Med. 1991;14:1412-17, Wong et al., Ophthalmology. 2008;115:116-26).

VEGF has been shown to be elevated in patients with nAMD and is thought to play a key role in the neovascularization process (Spilsbury et al., Am J Pathol. 2000;157:135-44). The use of intravitreal (IVT) pharmacotherapy targeting VEGF has significantly improved visual outcomes in patients with nAMD (Bloch et al., Am J Ophthalmol. 2012;153:209-13, Campbell et al., Arch Ophthalmol. 2012;130:794-5). Anti-VEGF treatments, such as ranibizumab (LUCENTIS®), aflibercept (EYLEA®), and brolucizumab (Beovu®), inhibit VEGF signaling pathways and have been shown to halt the growth of neovascular lesions and resolve retinal edema.

In two Phase 3 studies of ranibizumab, with monthly dosing regimens, approximately 95% of ranibizumab treated subjects experienced stabilization of vision (defined as a loss of fewer than 15 ETDRS letters) or improvement in vision at 12 months compared with 62% and 64% in the control groups (Rosenfeld et al., N Engl J Med. 2006;355:1419-31, Brown et al., N Engl J Med. 2006;355:1432-44). Twenty-five to 40% of subjects in the ranibizumab groups gained ≥15 letters at 12 months compared with 5-6% in the 2 control groups. On average, ranibizumab treated subjects gained 7-11 letters of vision after 12 months, whereas control subjects lost an average of approximately 10 letters. This gain in visual acuity was essentially maintained during the second year of both Phase 3 studies while vision, on average, continued to decline in the control group. The visual acuity benefits, which indicate a suspension of nAMD rather than a slowdown of its progression, were supported by corresponding effects on lesion anatomy and subject reported outcomes. The latter demonstrated statistically and clinically meaningful improvements in near activities, distance activities, and vision specific dependency as measured by the National Eye Institute Visual Functioning Questionnaire-25 (VFQ-25).

In two parallel Phase 3 trials of aflibercept, treatment naïve subjects with nAMD were randomized to 2 doses (0.5 and 2.0 mg) and 2 regimen (every 4 weeks and every 8 weeks with 2.0 mg) or the control arm (ranibizumab 0.5 mg every 4 weeks). At 52 weeks, all aflibercept groups, independent of doses and regimen, were noninferior to the ranibizumab group with equal maintenance of vision in 95% of eyes (Heier et al., Ophthalmology. 2012;119:2537-48). In the 2 mg aflibercept every 4 weeks group, there was a mean BCVA improvement of 9.3 letters and in the 2 mg aflibercept every 8 weeks group there was an improvement of 8.4 letters compared to the control group which had a mean improvement of 8.7 letters. In the second year of the study subjects were switched to a capped pro-re-nata (PRN) regimen. The proportion of subjects who maintained BCVA ranged between 91% and 92% for all groups. Mean BCVA improvements ranged from 7.9 (ranibizumab 0.5 mg every 4 weeks), 7.6 (aflibercept 2 mg every 4 weeks and every 8 weeks) to 6.6 (aflibercept 0.5 mg). Over all groups, a mean loss of 0.8-1.7 letters was seen after switching from a fixed to a capped PRN regimen. The retreatment frequency was similar between aflibercept and ranibizumab arms during the capped PRN year, with 4.1 injections for the aflibercept 2 mg every 4 weeks arm, 4.2 injections for the aflibercept 2 mg every 8 weeks arm and 4.7 for the ranibizumab 0.5 mg every 4 weeks arm (Schmidt-Erfurth et al., Br J Ophthalmol 2014;98:1144-1167 2014;98:1144-1167).

Two similarly designed phase 3 trials (HAWK and HARRIER) compared brolucizumab, a single-chain antibody fragment that inhibits vascular endothelial growth factor-A, with aflibercept to treat nAMD (Dugel et al., Ophthalmology, Volume 127, Issue 1, January 2020, Pages 72-84). The HAWK (NCT02307682) and HARRIER (NCT02434328) studies, which were 2-year, double-masked, multicenter Phase 3 studies investigating the efficacy of brolucizumab versus aflibercept in treatment naïve patients with nAMD. Patients were randomized to intravitreal brolucizumab 3 mg (HAWK only) or 6 mg or aflibercept 2 mg. After loading with 3 monthly injections, brolucizumab-treated eyes received an injection every 12 weeks (q12w) and were interval adjusted to every 8 weeks (q8w) if disease activity was present; aflibercept-treated eyes received q8w dosing. Brolucizumab was noninferior to aflibercept in visual function at Week 48, and >50% of brolucizumab 6 mg-treated eyes were maintained on q12w dosing interval through Week 48. Anatomic outcomes favored brolucizumab over aflibercept. Clinical treatment-effect data from patients receiving brolucizumab 6 mg in the HAWK and HARRIER studies were also compared with modelled placebo data (Agostini et al., Curr Eye Res, 2020 Oct;45(10):1298-1301). Compared with a modelled placebo, brolucizumab treatment was associated with an overall best corrected visual acuity gain of approximately 22 Early Treatment Diabetic Retinopathy Study (ETDRS) letters at Week 48 and 28 letters at Week 96.

Currently marketed anti-VEGF treatments typically start with a loading phase of 3 monthly doses, followed by maintenance dosing, either with fixed (e.g. every 4 or 8 weeks or every 12 weeks) or individualized treatment intervals, based on pro re nata (PRN) or Treat-and-Extend (T&E) concepts (Wykoff et al., 2018). Monthly treatment or treatment every 2months poses significant burden not only for the generally older patients but also for their caregivers and physicians. Also, although the treatments have proven to have a positive benefit/risk ratio, they are not without risk. Each injection carries with it the possibility of pain, sub-conjunctival hemorrhage, vitreous hemorrhage, retinal tear, retinal detachment, iatrogenic cataract, and endophthalmitis (Ohr et al., Expert Opin. Pharmacother. 2012;13:585-591), as well as a sustained rise in intraocular pressure (IOP) with serial injections of anti-VEGF agents (Tseng et al., J Glaucoma. 2012;21:241-47). Additionally, even with monthly IVT injections, 60-70% of patients gain less than 15 letters of visual acuity. In ranibizumab and aflibercept trials, both interventional (e.g. TREND (Silva et al., Ophthalmology; 2018, 125:57-65), ALTAIR (Bayer AG, 2017, Package leaflet Eylea®-Germany)) and real life studies (prospective non-interventional trials, e.g. OCEAN (Voegeler and Mueller, Non-interventional Final Study Report CRFB002ADE18, 2017)), when extended to q6w or longer treatment intervals, a number of patients still showed persistent fluid, although initial functional and anatomical response after the loading phase (three initial injections) was seen. For these patients, a longer lasting anti-VEGF agent like brolucizumab (e.g. maintenance dosing every 8 or 12 weeks) may lead to optimized fluid, and disease control i.e. sustained functional and anatomical response, respectively; which overall might result in improved patient care (e.g. less frequent visits, reduced treatment burden).

Despite the treatment success of existing anti-VEGFs, there remains a need for further treatment options to improve response rate and/or reduce resource use and injection frequency in patients with nAMD. Current dosing regimens include a loading phase of three monthly doses. There is a medical need to improve and optimize the dosing regimen for anti-VEGF in nAMD patients, in particular, the loading phase dosing regimens, to improve patient care (e.g. less frequent visits, reduced treatment burden).

### SUMMARY

The invention provides a method for treating neovascular age-related macular degeneration (nAMD) in a patient, the method comprises administering to the patient as a loading phase of two or three individual doses of a VEGF antagonist at 6-week interval (q6w regimen), and followed by administering to the patient one or more additional doses of the VEGF antagonist, wherein each additional dose is administered in an administration interval of at least 8 weeks after the immediately preceding dose, e.g., once every 8 weeks (q8w regimen) to once every 12 weeks (q12w regimen), e.g., once every 8 weeks (q8w regimen) or once every 12 weeks (q12w regimen).

Currently marketed anti-VEGF treatments typically start with a loading phase of 3 monthly doses, followed by maintenance dosing, either with fixed (e.g. every 4 or 8 weeks or every 12 weeks) or individualized treatment intervals, based on pro re nata (PRN) or Treat-and-Extend (T&E) concepts (Wykoff et al., 2018). Each injection carries with it the possibility of pain, sub-conjunctival hemorrhage, vitreous hemorrhage, retinal tear, retinal detachment, iatrogenic cataract, and endophthalmitis (Ohr et al., Expert Opin. Pharmacother. 2012;13:585-591), as well as a potential for sustained rise in intraocular pressure (IOP) with serial injections of anti-VEGF agents (Tseng et al., J Glaucoma. 2012;21:241-47). The inventors have now performed a quantitative systems pharmacology model to simulate intraocular brolucizumab PK and VEGF inhibition for alternative dosing regimens. The results of the Beovu arms of the HAWK and HARRIER studies, where Beovu was administered every 4 weeks (monthly) for the first 3 doses followed by dosing every 12 or 8 weeks (q12w/q8w), were replicated in a population PK/PD model simulation study where Beovu was administered every 6 weeks (q6w) for the first 2 doses followed by dosing every 12 or 8 weeks (q12w/q8w). The inventors have surprisingly found that a reduced dose intensity within the loading period, namely two or three individual doses of a VEGF antagonist at 6-week interval, may still maintain robust VEGF inhibition as the approved three-dose loading regimen (3× Q4W followed by maintenance doses) while reducing the treatment burden and associated risks.

In one aspect, the present invention provides a method for treating nAMD in a patient, the method comprises: (a) administering to the patient as a loading phase of two individual doses of a VEGF antagonist at 6-week interval (q6w regimen), and (b) assessing the patient for disease activity after the second dose of the loading phase, and optionally, wherein if presence of disease activity is identified after the second dose of the VEGF antagonist, a third dose of the VEGF antagonist is administered to the patient 6 weeks after the second dose as part of the loading phase.

In a further aspect, the present invention provides a VEGF antagonist for use as a medicament for treating nAMD in a patient, wherein the VEGF antagonist is administered to the patient as two individual doses at 6-week interval (q6w regimen) in a loading phase, followed by assessing the patient for disease activity after the second dose of the loading phase, and optionally, wherein if presence of disease activity is identified after the second dose of the VEGF antagonist, a third dose of the VEGF antagonist is administered to the patient 6 weeks after the second dose as part of the loading phase.

In another aspect, the present invention provides a pharmaceutical composition comprising a VEGF antagonist for use as a medicament for treating nAMD in a patient, wherein the pharmaceutical composition is administered to the patient as two individual doses at 6-week interval (q6w regimen) in a loading phase, followed by assessing the patient for disease activity after the second dose of the loading phase, and optionally, wherein if presence of disease activity is identified after the second dose of the pharmaceutical composition, a third dose of the pharmaceutical composition is administered to the patient 6 weeks after the second dose as part of the loading phase.

In another aspect, the present invention provides use of a VEGF antagonist for the manufacture of a medicament for treating nAMD in a patient, wherein the use comprises (a) administering to the patient as a loading phase of two individual doses of a VEGF antagonist at 6-week interval (q6w regimen), and (b) assessing the patient for disease activity after the second dose of the loading phase, and (c) optionally, if presence of disease activity is identified after the second dose of the VEGF antagonist, administering a third dose of the VEGF antagonist to the patient 6 weeks after the second dose as part of the loading phase.

In one embodiment, the methods and uses of the present invention further comprise administering to the patient after the loading phase a maintenance phase of one or more additional individual doses of the VEGF antagonist, wherein each additional dose is administered at an administration interval of at least once every 8 weeks (q8w regimen), e.g., once every 8 weeks (q8w regimen) to once every 12 weeks (q12w regimen), e.g., once every 8 weeks (q8w regimen) or once every 12 weeks (q12w regimen).

In one aspect, the present invention provides a method for treating nAMD in a patient, the method comprises:
(a) administering to the patient two individual doses of a VEGF antagonist at 6-week interval (q6w regimen); and
(b) optionally, assessing the patient for disease activity after the second dose of the VEGF antagonist; and
(c) optionally, if presence of disease activity is identified after the second dose of the VEGF antagonist, administering to the patient a third dose of the VEGF antagonist 6 weeks after the administration of the second dose (q6w regimen), and
(d) administering to the patient one or more additional doses of the VEGF antagonist, wherein each additional dose is administered in an administration interval of at least 8 weeks after the immediately preceding dose, e.g., once every 8 weeks (q8w regimen) to once every 12 weeks (q12w regimen), e.g., once every 8 weeks (q8w regimen) or once every 12 weeks (q12w regimen).

In a further aspect, the present invention provides a VEGF antagonist for use as a medicament for treating nAMD in a patient, wherein:
(a) the VEGF antagonist is administered to the patient as two individual doses at 6-week intervals (q6w regimen);
(b) optionally, followed by assessing the patient for disease activity after the second dose of the VEGF antagonist; and optionally, if presence of disease activity is identified after the second dose of the VEGF antagonist, a third dose of the VEGF antagonist is administered to the patient 6 weeks after the administration of the second dose (q6w regimen);
(c) followed by one or more additional doses, wherein each additional dose is administered in an administration interval of at least 8 weeks after the immediately preceding dose, e.g., once every 8 weeks (q8w regimen) to once every 12 weeks (q12w regimen), e.g., once every 8 weeks (q8w regimen) or once every 12 weeks (q12w regimen).

In a further aspect, the present invention provides a pharmaceutical composition comprising a VEGF antagonist for use as a medicament for treating nAMD in a patient, wherein:
(a) the pharmaceutical composition is administered to the patient as two individual doses at 6-week intervals (q6w regimen);
(b) optionally, followed by assessing the patient for disease activity after the second dose of the pharmaceutical composition; and optionally, if presence of disease activity is identified after the second dose of the pharmaceutical composition, a third dose of the pharmaceutical composition is administered to the patient 6 weeks after the administration of the second dose (q6w regimen);
(c) followed by one or more additional doses, wherein each additional dose is administered in an administration interval of at least 8 weeks after the immediately preceding dose, e.g., once every 8 weeks (q8w regimen) to once every 12 weeks (q12w regimen), e.g., once every 8 weeks (q8w regimen) or once every 12 weeks (q12w regimen).

In another aspect, the present invention provides use of a VEGF antagonist for the manufacture of a medicament for treating nAMD in a patient, the use comprising:
(a) administering to the patient two individual doses of the VEGF antagonist at 6-week interval (q6w regimen);
(b) optionally, followed by assessing the patient for disease activity after the second dose of the VEGF antagonist; and optionally, if presence of disease activity is identified after the second dose of the VEGF antagonist, administering a third dose of the VEGF antagonist 6 weeks after the administration of the second dose (q6w regimen);
(c) administering to the patient one or more additional doses of the VEGF antagonist, wherein each additional dose is administered in an administration interval of at least 8 weeks after the immediately preceding dose, e.g., once every 8 weeks (q8w regimen) to once every 12 weeks (q12w regimen), e.g., once every 8 weeks (q8w regimen) or once every 12 weeks (q12w regimen).

In certain embodiments, the VEGF antagonist used in the methods and the uses of the invention is an anti-VEGF antibody, in a particular wherein the anti-VEGF antibody is a single chain antibody (scFv) or Fab fragment. In certain embodiments, the VEGF antagonist used in the methods and the uses of the invention comprises the sequences of SEQ ID NO:1 and SEQ ID NO:2, more particularly wherein the anti-VEGF antibody is brolucizumab. In certain embodiments, the methods and the uses of the invention comprise administering to the patient one or more doses of the VEGF antagonist, wherein the VEGF antagonist is brolucizumab and the dose of the VEGF antagonist is about 3 mg to about 6 mg, in particular about 3 mg or about 6 mg, more particularly 6 mg.

Non-limiting embodiments of the present disclosure are described in the following embodiments:
Embodiment 1: A method for treating nAMD in a patient, the method comprising:
(a) administering to the patient as a loading phase of two individual doses of a VEGF antagonist at 6-week interval (q6w regimen), and
(b) assessing the patient for disease activity after the second dose of the loading phase, e.g., assessing the patient for disease activity between ≥0 and ≤6 weeks after the second dose of the loading phase.
Embodiment 2: The method of embodiment 1, wherein if presence of disease activity is identified after the second dose of the VEGF antagonist, the method further comprises administering to the patient as part of the loading phase a third dose of the VEGF antagonist 6 weeks after the second dose.
Embodiment 3: The method of embodiment 1 or 2, comprising administering to the patient after the loading phase a maintenance phase of one or more additional individual doses of the VEGF antagonist, wherein each additional dose is administered at an administration interval of at least once every 8 weeks (q8w regimen), e.g., once every 8, 9, 10, 11, 12, 13, 14, 15, 16, 17, 18, 19, 20, 21, 22, 23, or 24 weeks, e.g., once every 8 weeks (q8w regimen) to once every 12 weeks (q12w regimen), e.g., once every 8 weeks (q8w regimen) or once every 12 weeks (q12w regimen).
Embodiment 4: The method of embodiment 1 or 2, comprising administering to the patient after the loading phase a maintenance phase of one or more additional individual doses of the VEGF antagonist, wherein each additional dose is administered at an administration interval of at least once every 12 weeks (q12w regimen).
Embodiment 5: The method of any one of the preceding embodiments, comprising assessing the patient for disease activity during the maintenance phase and administering to the patient additional doses at an administration interval of once every 8 weeks (q8w regimen) when there is disease activity observed and administering to the patient additional doses at an administration interval of once every 12 weeks (q12w regimen) when there no disease activity observed.
Embodiment 6: A method for treating nAMD in a patient, the method comprising:
(a) administering to the patient two individual doses of a VEGF antagonist at 6-week interval (q6w regimen); and
(b) administering to the patient one or more additional doses of the VEGF antagonist, wherein each additional dose is administered in an administration interval of at least 8 weeks after the immediately preceding dose, e.g., 8, 9, 10, 11, 12, 13, 14, 15, 16, 17, 18, 19, 20, 21, 22, 23, or 24 weeks, e.g., once every 8 weeks (q8w regimen) to once every 12 weeks (q12w regimen), e.g., once every 8 weeks (q8w regimen) or once every 12 weeks (q12w regimen).
Embodiment 7: The method of embodiment 6, wherein the method comprises:
(a) administering to the patient two individual doses of a VEGF antagonist at 6-week interval (q6w regimen); and
(b) assessing the patient for disease activity after the second dose of the VEGF antagonist, e.g., assessing the patient for disease activity between ≥0 and ≤6 weeks after the second dose of the VEGF antagonist; and
(c) administering to the patient one or more additional doses of the VEGF antagonist, wherein each additional dose is administered in an administration interval of at least 8 weeks after the immediately preceding dose, e.g., 8, 9, 10, 11, 12, 13, 14, 15, 16, 17, 18, 19, 20, 21, 22, 23, or 24 weeks, e.g., once every 8 weeks (q8w regimen) to once every 12 weeks (q12w regimen), e.g., once every 8 weeks (q8w regimen) or once every 12 weeks (q12w regimen).
Embodiment 8: The method of embodiment 7, wherein if presence of disease activity is identified after the second dose of the VEGF antagonist, the method further comprises administering to the patient a third dose of the VEGF antagonist 6 weeks after the administration of the second dose (q6w regimen).
Embodiment 9: The method of any one of the preceding embodiments, wherein the method does not comprise administering to the patient more than 3 doses in an administration interval of less than 8 week, e.g., wherein the method does not comprise administering to the patient more than 3 doses in an administration interval of 6 weeks.
Embodiment 10: The method of any one of the preceding embodiments, wherein the method further comprises assessing the patient for disease activity before or after administering every q8w or q12w dose of the VEGF antagonist.
Embodiment 11: The method of embodiment 10, wherein if presence of disease activity is identified after a q12w dose of the VEGF antagonist, the patient is switched to a q8w regimen of the VEGF antagonist.
Embodiment 12: The method of any one of embodiments 1 to 5 or 7 to 11, wherein the disease activity is assessed based on one or more of the following: best corrected visual acuity (BCVA), visual acuity (VA), central subfield thickness (CSFT), and/or presence of intraretinal cysts/fluid.
Embodiment 13: The method of embodiment 12, wherein the presence of disease activity includes one or more of the following: (i) decrease in Best Corrected Visual Acuity (BCVA), (ii) decrease in Visual Acuity (VA), (iii) increase or lack of reduction in Central Subfield Thickness (CSFT), (iv) new or persistent or recurrent Intraretinal Cysts (IRC) and/or Intraretinal Fluid (IRF) and/or Subretinal Fluid (SRF).
Embodiment 14: The method of any one of preceding embodiments, wherein the VEGF antagonist is an anti-VEGF antibody, e.g., a single chain antibody (scFv) or Fab fragment.
Embodiment 15: The method of any one of preceding embodiments, wherein the anti-VEGF antagonist comprises the sequences of SEQ ID NO: 1 and SEQ ID NO:2.
Embodiment 16: The method of embodiment 12, wherein the VEGF antagonist is an anti-VEGF antibody comprising the sequence of SEQ ID NO: 3 or SEQ ID NO: 4.
Embodiment 17: The method of embodiment 12 or 13, wherein the anti-VEGF antagonist is brolucizumab.
Embodiment 18: The method of any one of preceding embodiments wherein the VEGF antagonist is administered by an injection, e.g., intravitreal injection.
Embodiment 19: The method of any one of preceding embodiments wherein the dose of the VEGF antagonist is from about 3 mg to about 6 mg, e.g., about 3 mg or about 6 mg, e.g., 6 mg.
Embodiment 20: The method of any one of preceding embodiments, wherein the patient is a human.
Embodiment 21: A VEGF antagonist for use as a medicament for treating nAMD in a patient, wherein the VEGF antagonist is administered to the patient as two individual doses at 6-week interval (q6w regimen) in a loading phase, followed by assessing the patient for disease activity after the second dose of the loading phase, e.g., assessing the patient for disease activity between ≥0 and ≤6 weeks after the second dose of the loading phase, and optionally, wherein if presence of disease activity is identified after the second dose of the VEGF antagonist, a third dose of the VEGF antagonist is administered to the patient 6 weeks after the second dose as part of the loading phase.
Embodiment 22: The VEGF antagonist for use of embodiment 21, wherein, after the loading phase, one or more additional individual doses of the VEGF antagonist are administered to the patient as a maintenance phase, wherein each additional dose is administered at an administration interval of at least once every 8 weeks (q8w regimen), e.g., once every 8, 9, 10, 11, 12, 13, 14, 15, 16, 17, 18, 19, 20, 21, 22, 23, or 24 weeks, e.g., once every 8 weeks (q8w regimen) to once every 12 weeks (q12w regimen), e.g., once every 8 weeks (q8w regimen) or once every 12 weeks (q12w regimen).
Embodiment 23: A pharmaceutical composition comprising a VEGF antagonist for use as a medicament for treating nAMD in a patient, wherein the pharmaceutical composition is administered to the patient as two individual doses at 6-week interval (q6w regimen) in a loading phase, followed by assessing the patient for disease activity after the second dose of the loading phase, e.g., assessing the patient for disease activity between ≥0 and ≤6 weeks after the second dose of the loading phase, and optionally, wherein if presence of disease activity is identified after the second dose of the pharmaceutical composition, a third dose of the pharmaceutical composition is administered to the patient 6 weeks after the second dose as part of the loading phase.
Embodiment 24: The pharmaceutical composition for use of embodiment 23, wherein, after the loading phase, one or more additional individual doses of the pharmaceutical composition are administered to the patient as a maintenance phase, wherein each additional dose is administered at an administration interval of at least once every 8 weeks (q8w regimen), e.g., once every 8, 9, 10, 11, 12, 13, 14, 15, 16, 17, 18, 19, 20, 21, 22, 23, or 24 weeks, e.g., once every 8 weeks (q8w regimen) to once every 12 weeks (q12w regimen), e.g., once every 8 weeks (q8w regimen) or once every 12 weeks (q12w regimen).
Embodiment 25: Use of a VEGF antagonist for the manufacture of a medicament for treating nAMD in a patient, wherein the use comprises
(a) administering to the patient as a loading phase of two individual doses of a VEGF antagonist at 6-week interval (q6w regimen), and
(b) assessing the patient for disease activity after the second dose of the loading phase, e.g., assessing the patient for disease activity between ≥0 and ≤6 weeks after the second dose of the loading phase, and
(c) optionally, if presence of disease activity is identified after the second dose of the VEGF antagonist, the use further comprises administering to the patient as part of the loading phase a third dose of the VEGF antagonist 6 weeks after the second dose.
Embodiment 26: The use of embodiment 25, wherein the use further comprises administering to the patient after the loading phase a maintenance phase of one or more additional individual doses of the VEGF antagonist, wherein each additional dose is administered at an administration interval of at least once every 8 weeks (q8w regimen), e.g., once every 8, 9, 10, 11, 12, 13, 14, 15, 16, 17, 18, 19, 20, 21, 22, 23, or 24 weeks, e.g., once every 8 weeks (q8w regimen) to once every 12 weeks (q12w regimen), e.g., once every 8 weeks (q8w regimen) or once every 12 weeks (q12w regimen).
Embodiment 27: A VEGF antagonist for use as a medicament for treating nAMD in a patient, wherein:
(a) the VEGF antagonist is administered to the patient as two individual doses at 6-week intervals (q6w regimen);
(b) optionally, followed by assessing the patient for disease activity after the second dose of the VEGF antagonist, e.g., assessing the patient for disease activity between ≥0 and ≤6 weeks after the second dose of the VEGF antagonist; and optionally, if presence of disease activity is identified after the second dose of the VEGF antagonist, a third dose of the VEGF antagonist is administered to the patient 6 weeks after the administration of the second dose (q6w regimen);
(c) followed by one or more additional doses, wherein each additional dose is administered in an administration interval of at least 8 weeks after the immediately preceding dose, e.g., 8, 9, 10, 11, 12, 13, 14, 15, 16, 17, 18, 19, 20, 21, 22, 23, or 24 weeks, e.g., once every 8 weeks (q8w regimen) to once every 12 weeks (q12w regimen), e.g., once every 8 weeks (q8w regimen) or once every 12 weeks (q12w regimen).

Embodiment 28: A pharmaceutical composition comprising a VEGF antagonist for use as a medicament for treating nAMD in a patient, wherein:
(a) the pharmaceutical composition is administered to the patient as two individual doses at 6-week intervals (q6w regimen);
(b) optionally, followed by assessing the patient for disease activity after the second dose of the pharmaceutical composition, e.g., assessing the patient for disease activity between ≥0 and ≤6 weeks after the second dose of the pharmaceutical composition; and optionally, if presence of disease activity is identified after the second dose of the pharmaceutical composition, a third dose of the pharmaceutical composition is administered to the patient 6 weeks after the administration of the second dose (q6w regimen);
(c) followed by one or more additional doses, wherein each additional dose is administered in an administration interval of at least 8 weeks after the immediately preceding dose, e.g., 8, 9, 10, 11, 12, 13, 14, 15, 16, 17, 18, 19, 20, 21, 22, 23, or 24 weeks, e.g., once every 8 weeks (q8w regimen) to once every 12 weeks (q12w regimen), e.g., once every 8 weeks (q8w regimen) or once every 12 weeks (q12w regimen).
Embodiment 29: Use of a VEGF antagonist for the manufacture of a medicament for treating nAMD in a patient, the use comprising:
(a) administering to the patient two individual doses of the VEGF antagonist at 6-week interval (q6w regimen);
(b) optionally, followed by assessing the patient for disease activity after the second dose of the VEGF antagonist, e.g., assessing the patient for disease activity between ≥0 and ≤6 weeks after the second dose of the VEGF antagonist; and optionally, if presence of disease activity is identified after the second dose of the VEGF antagonist, administering a third dose of the VEGF antagonist 6 weeks after the administration of the second dose (q6w regimen); and
(c) administering to the patient one or more additional doses of the VEGF antagonist, wherein each additional dose is administered in an administration interval of at least 8 weeks after the immediately preceding dose, e.g., 8, 9, 10, 11, 12, 13, 14, 15, 16, 17, 18, 19, 20, 21, 22, 23, or 24 weeks, e.g., once every 8 weeks (q8w regimen) to once every 12 weeks (q12w regimen), e.g., once every 8 weeks (q8w regimen) or once every 12 weeks (q12w regimen).

Specific preferred embodiments of the invention will become evident from the following more detailed description of certain preferred embodiments and the claims.

### BRIEF DESCRIPTION OF THE DRAWINGS

FIG. 1. Retinal free drug and VEGF concentrations for 3× Q4W loading phase followed by Q8W maintenance phase.

FIG. 2. Retinal VEGF concentrations for 3× Q4W loading and 2× Q6W loading phases.

FIG. 3. Retinal VEGF concentrations for 3× Q4W and 2× Q6W loading phases followed by Q8W and Q12W maintenance phases.

FIG. 4. Simulated CSFT change from baseline during first year of aflibercept 2 mg treatment of HAWK and HARRIER studies.

FIG. 5. Simulated BCVA change from baseline during first year of aflibercept 2 mg treatment of HAWK and HARRIER studies.

FIG. 6. Illustration of disease activity presence based on CSFT.

FIG. 7. Week 48 percent of patients on q12w at various thresholds in simulated brolucizumab 6 mg treatment arms of HAWK and HARRIER.

FIG. 8. Mean simulated and observed CSFT change from baseline after 3 q4w loading injections and individualized q12w/q8w treatment.

FIG. 9. Mean simulated and observed BCVA change from baseline after 3 q4w loading injections and individualized q12w/q8w treatment.

FIG. 10. Schematics of simulated DA assessments and treatment schedule.

FIG. 11. Mean CSFT change from baseline after simulated q6w loading and individualized q12w/q8w treatment or from observed data.

FIG. 12. Mean BCVA change from baseline after simulated q6w loading and individualized q12w/q8w treatment or from observed data.

### DETAILED DESCRIPTION

### Definitions

Unless defined otherwise, all technical and scientific terms used herein have the same meaning as commonly understood by those of ordinary skill in the art to which this present disclosure pertains. Any references cited herein, including, e.g., all patents, published patent applications, and non-patent publications, are incorporated by reference in their entirety. To facilitate understanding of the disclosure, several terms and abbreviations as used herein are defined below as follows:

As used herein and unless otherwise indicated, the terms “a” and “an” are taken to mean “one”, “at least one” or “one or more”. Unless otherwise required by context, singular terms used herein shall include pluralities and plural terms shall include the singular.

As used herein, the term “about” includes and describes the value or parameter per se. For example, “about x” includes and describes “x” per se. As used herein, the term “about” when used in association with a measurement, or used to modify a value, a unit, a constant, or a range of values, refers to variations of ±1-10% in addition to including the value or parameter per se. In some embodiments, the term “about” when used in association with a measurement, or used to modify a value, a unit, a constant, or a range of values, refers to variations of ±1, ±2, ±3, ±4, ±5, ±6, ±7, ±8, ±9, or ±10%.

The term “VEGF” refers to the 165-amino acid vascular endothelial cell growth factor, and related 121-, 189-, and 206-amino acid vascular endothelial cell growth factors, as described by Leung et al., Science 246:1306 (1989), and Houck et al., Mol. Endocrin. 5:1806 (1991) together with the naturally occurring allelic and processed forms of those growth factors. The term “VEGF”, in particular, refers to the human VEGF.

The term “VEGF receptor” or “VEGFr” refers to a cellular receptor for VEGF, ordinarily a cell-surface receptor found on vascular endothelial cells, as well as variants thereof retaining the ability to bind hVEGF. One example of a VEGF receptor is the fms-like tyrosine kinase (flt), a transmembrane receptor in the tyrosine kinase family. DeVries et al., Science 255:989 (1992); Shibuya et al., Oncogene 5:519 (1990). The flt receptor comprises an extracellular domain, a transmembrane domain, and an intracellular domain with tyrosine kinase activity. The extracellular domain is involved in the binding of VEGF, whereas the intracellular domain is involved in signal transduction. Another example of a VEGF receptor is the flk-1 receptor (also referred to as KDR). Matthews et al., Proc. Nat. Acad. Sci. 88:9026 (1991); Terman et al., Oncogene 6:1677 (1991); Terman et al., Biochem. Biophys. Res. Commun. 187:1579 (1992). Binding of VEGF to the flt receptor results in the formation of at least two high molecular weight complexes, having an apparent molecular weight of 205,000 and 300,000 Daltons. The 300,000 Dalton complex is believed to be a dimer comprising two receptor molecules bound to a single molecule of VEGF.

As used herein, a “VEGF antagonist” refers to a compound that can diminish or inhibit VEGF activity in vivo. A VEGF antagonist can bind to a VEGF receptor(s) or block VEGF protein(s) from binding to VEGF receptor(s). A VEGF antagonist can be, for example, a small molecule, an anti-VEGF antibody or antigen-binding fragments thereof, fusion protein (such as aflibercept or other such soluble decoy receptor), an aptamer, an antisense nucleic acid molecule, an interfering RNA, receptor proteins, and the like that can bind specifically to one or more VEGF proteins or one or more VEGF receptors. Several VEGF antagonists are described in WO 2006/047325. In one embodiment, the VEGF antagonist is any licensed anti-VEGF drug such as brolucizumab, ranibizumab or aflibercept. In one embodiment, the VEGF antagonist is an anti-VEGF antibody (such as brolucizumab or ranibizumab or bevacizumab or a bi-specific antibody such as faricimab) or an anti-VEGF DARPin (such as abicipar) or a soluble VEGF receptor (e.g., a fusion protein composed of the VEGF receptor domains, such as a fusion protein composed of the combination between VEGF receptor domains with the Fc fragment of human immunoglobulin with the Fc fragment of human immunoglobulin, e.g., conbercept, aflibercept) or AAV containing a sequence encoding for an anti-VEGF antibody (such as RGX-314 from Regenxbio), or AAV containing a sequence encoding the VEGF receptor domains, e.g., conbercept (such as ADVM-022 from Adverum) or any licensed anti-VEGF drug (such as brolucizumab, ranibizumab or aflibercept).

The term “antibody” as used herein includes whole antibodies and any antigen binding fragment (i.e., “antigen-binding portion”, “antigen binding polypeptide”, or “immunobinder”) or single chain thereof. An “antibody” includes a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “single chain antibody”, “single chain Fv” or “scFv” is intended to refer to a molecule comprising an antibody heavy chain variable domain (or region; V_{H}) and an antibody light chain variable domain (or region; V_{L}) connected by a linker. Such scFv molecules can have the general structures: NH2-VL-linker-VH-COOH or NH2-VH-linker-VL-COOH.

The term “antigen-binding portion” of an antibody (or simply “antibody portion”) refers to one or more fragments of an antibody that retain the ability to specifically bind to an antigen (e.g., VEGF). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a single domain or dAb fragment (Ward et al., (1989) Nature 341:544-546), which consists of a VH domain; and (vi) an isolated complementarity determining region (CDR) or (vii) a combination of two or more isolated CDRs which may optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins. Antibodies can be of different isotype, for example, an IgG (e.g., an IgG1, IgG2, IgG3, or IgG4 subtype), IgA1, IgA2, IgD, IgE, or IgM antibody.

As used herein, the term “subject” or “patient” refers to human and non-human mammals, including but, not limited to, primates, pigs, horses, dogs, cats, sheep, and cows. As used herein, a “mammal” includes any animal classified as a mammal, including, but not limited to, humans, domestic animals, farm animals, and companion animals, etc.

The term “treat”, “treating” or “treatment” includes therapeutic treatments, prophylactic treatments and applications in which one reduces the risk that a subject will develop a disorder or other risk factor. Treatment does not require the complete curing of a disorder and encompasses the reduction of the symptoms or underlying risk factors. As used herein, the terms “treat”, “treatment” and “treating” refer to the reduction or amelioration of the progression or severity of neovascular (exudative) age-related macular degeneration (nAMD) or the amelioration of one or more symptoms, suitably of one or more discernible symptoms of nAMD. In specific embodiments, the terms “treat”, “treatment” and “treating” refer to the amelioration of at least one measurable physical parameter of nAMD (such as achieve or at least partially achieve a desired effect (e.g. the partial or complete regression of retinal neovascularization, decrease of retinal fluid or achieving retinal fluid-free status, e.g., intraretinal fluid (IRF) and subretinal fluid (SRF), decrease of Central Subfield Thickness (CSFT), improvement in vision, e.g., a change of BCVA>1, >2, >3, >4 or >5 letters, or a DRSS score <61), wherein the physical parameter is not necessarily discernible by the patient.

The term “loading phase” refers to the first 2 or 3 doses of a VEGF antagonist administered at q6w intervals. Amount of doses in a loading phase (2 or 3 doses) can be adjusted based on Disease Activity Assessments as described herein.

The term “maintenance phase” refers to additional doses at ≥8 weeks intervals, e.g., 8, 9, 10, 11, 12, 13, 14, 15, 16, 17, 18, 19, 20, 21, 22, 23, or 24 weeks intervals, and can be adjusted based on Disease Activity Assessments as described herein. Suitably, the term “maintenance phase” refers to additional doses once every 8 weeks (q8w regimen) to once every 12 weeks (q12w regimen), e.g., once every 8 weeks (q8w regimen) or once every 12 weeks (q12w regimen), and can be adjusted based on Disease Activity Assessments as described herein.

As used herein, an administration interval can be referred to as qXw, where the “X” is a number of weeks between administered doses. For example, q6w is an interval of 6 weeks.

As used herein, the term “week” means 7 days±1 day. As used herein, the term “month” means 25 to 31 days. Also, as used herein, the term “month” means 4 weeks.

As used herein, the terms “dose” or “therapeutically effective dose” refer to an amount of a therapy (e.g., a VEGF antagonist, e.g., brolucizumab, or a pharmaceutical composition provided herein) which is sufficient to reduce and/or ameliorate the severity of a given condition, disorder, or disease and/or a symptom related thereto. The term “dose” or “therapeutically effective dose” is defined as an amount sufficient to achieve or at least partially achieve a desired effect (e.g. the partial or complete regression of retinal neovascularization, decrease of retinal fluid or achieving retinal fluid-free status, e.g., intraretinal fluid (IRF) and subretinal fluid (SRF), decrease of Central Subfield Thickness (CSFT), improvement in vision, e.g., a change of BCVA>1, >2, >3, >4 or >5 letters, or a DRSS score <61). A therapeutically effective dose is sufficient if it can produce even an incremental change in the symptoms or conditions associated with the disease. The therapeutically effective dose does not have to completely cure the disease or completely eliminate symptoms. Preferably, a therapeutically effective dose can at least partially arrest the disease and/or its complications in a patient already suffering from the disease. In specific embodiments, a therapeutic effective dose may involve repeated administration over a period of time. Amounts effective for this use will depend upon the severity of the disorder being treated and the general state of the patient's own immune system.

### Treatment Regimen

The invention provides methods for treating a patient having nAMD, the method comprising administering to the patient a VEGF antagonist on a treatment schedule that includes a loading phase and a maintenance phase as described herein. The invention provides a method for treating neovascular age-related macular degeneration (nAMD) in a patient, the method comprises administering to the patient as a loading phase of two or three individual doses of a VEGF antagonist at 6-week interval (q6w regimen), and followed by administering to the patient one or more additional doses of the VEGF antagonist, wherein each additional dose is administered in an administration interval of at least 8 weeks after the immediately preceding dose, e.g., once every 8 weeks (q8w regimen) to once every 12 weeks (q12w regimen), e.g., once every 8 weeks (q8w regimen) or once every 12 weeks (q12w regimen). In one aspect, the present invention provides a method for treating nAMD in a patient, the method comprising: (a) administering to the patient as a loading phase of two individual doses of a VEGF antagonist at 6-week interval (q6w regimen), and (b) assessing the patient for disease activity after the second dose of the loading phase. In certain embodiments, the present invention provides a method for treating nAMD in a patient, the method comprising: (a) administering to the patient as a loading phase of two individual doses of a VEGF antagonist at 6-week interval (q6w regimen), (b) assessing the patient for disease activity after the second dose of the loading phase, and (c) if presence of disease activity is identified after the second dose of the VEGF antagonist, administering to the patient as part of the loading phase a third dose of the VEGF antagonist 6 weeks after the second dose.

In a further aspect, the present invention provides a VEGF antagonist for use as a medicament or treating nAMD in a patient, wherein the VEGF antagonist is administered to the patient as two individual doses at 6-week interval (q6w regimen) in a loading phase, followed by assessing the patient for disease activity after the second dose of the loading phase. In certain embodiments, the present invention provides a VEGF antagonist for use as a medicament for treating nAMD in a patient, wherein: (a) the VEGF antagonist is administered to the patient as two individual doses at 6-week interval (q6w regimen) in a loading phase; (b) followed by assessing the patient for disease activity after the second dose of the loading phase; and (c) if presence of disease activity is identified after the second dose of the VEGF antagonist, a third dose of the VEGF antagonist is administered to the patient 6 weeks after the second dose as part of the loading phase.

In another aspect, the present invention provides a pharmaceutical composition comprising a VEGF antagonist for use as a medicament for treating nAMD in a patient, wherein the pharmaceutical composition is administered to the patient as two individual doses at 6-week interval (q6w regimen) in a loading phase, followed by assessing the patient for disease activity after the second dose of the loading phase. In certain embodiments, the present invention provides a pharmaceutical composition comprising a VEGF antagonist for use as a medicament for treating nAMD in a patient, wherein: (a) the pharmaceutical composition is administered to the patient as two individual doses at 6-week interval (q6w regimen) in a loading phase; (b) followed by assessing the patient for disease activity after the second dose of the loading phase; and (c) if presence of disease activity is identified after the second dose of the pharmaceutical composition, a third dose of the pharmaceutical composition is administered to the patient 6 weeks after the second dose as part of the loading phase.

In another aspect, the present invention provides use of a VEGF antagonist for the manufacture of a medicament for treating nAMD in a patient, wherein the use comprises (a) administering to the patient as a loading phase of two individual doses of the VEGF antagonist at 6-week interval (q6w regimen); and (b) assessing the patient for disease activity after the second dose of the loading phase. In certain embodiments, the present invention provides use of a VEGF antagonist for the manufacture of a medicament for treating nAMD in a patient, wherein the use comprises (a) administering to the patient as a loading phase of two individual doses of the VEGF antagonist at 6-week interval (q6w regimen); and (b) assessing the patient for disease activity after the second dose of the loading phase; and (c) if presence of disease activity is identified after the second dose of the VEGF antagonist, administering to the patient a third dose of the VEGF antagonist 6 weeks after the second dose as part of the loading phase.

In certain embodiments, the loading phase consists of two individual doses, administered at 6-week intervals (q6w), e.g., at day 0, and at week 6. In certain embodiments, the loading phase consists of three individual doses, administered at 6-week intervals (q6w), e.g., at day 0, at week 6, and at week 12. In certain embodiments, if presence of disease activity is identified after the second dose of the VEGF antagonist, e.g., if presence of disease activity is identified between ≥0 and ≤6 weeks after the second dose of the VEGF antagonist, a third dose of the VEGF antagonist is administered to the patient 6 weeks after the second dose as part of the loading phase.

In certain embodiments, the methods and uses of the present invention further comprises a maintenance phase as described herein. In certain embodiments, the methods and uses of the present invention further comprises administering to the patient after the loading phase a maintenance phase of one or more additional individual doses of the VEGF antagonist, wherein each additional dose is administered at an administration interval of at least once every 8 weeks (q8w regimen), e.g., once every 8, 9, 10, 11, 12, 13, 14, 15, 16, 17, 18, 19, 20, 21, 22, 23, or 24 weeks, e.g., once every 8 weeks (q8w regimen) to once every 12 weeks (q12w regimen), e.g., once every 8 weeks (q8w regimen) or once every 12 weeks (q12w regimen). In certain embodiments, the maintenance phase starts with a dosing regimen wherein the VEGF antagonist is administered once every 12-weeks (q12w), and the dosing interval is adjusted plus or minus 4-weeks depending on a disease activity assessment conducted before a dose is administered. In one embodiment, if disease activity is observed within 8 weeks after the last a q12w dose, the patient will receive the next dose 8 weeks (q8w dose) after the last q12w dose, thus being placed on a q8w dosing regimen until disease activity is no longer observed. In one embodiment, if disease activity is observed prior to administering a q12w dose, the patient will receive the q12w dose as planned, and receive the next dose 8 weeks later, thus being placed on a q8w dosing regimen until disease activity is no longer observed. When disease activity is no longer observed, the dosing regimen will be adjusted back to a q12w schedule. In another embodiment, if no disease activity is observed at any time during the maintenance phase, the treatment interval may be extended by 4 weeks, e.g., to a q16w. If disease activity is observed in a patient on a q16w dosing regimen, the treatment interval may be adjusted back to a q12w or q8w dosing regimen.

In one aspect, the present invention provides a method for treating nAMD in a patient, the method comprising: (a) administering to the patient two individual doses of a VEGF antagonist at 6-week interval (q6w regimen); and (b) administering to the patient one or more additional doses of the VEGF antagonist, wherein each additional dose is administered in an administration interval of at least 8 weeks after the immediately preceding dose, e.g., once every 8, 9, 10, 11, 12, 13, 14, 15, 16, 17, 18, 19, 20, 21, 22, 23, or 24 weeks, e.g., once every 8 weeks (q8w regimen) to once every 12 weeks (q12w regimen), e.g., once every 8 weeks (q8w regimen) or once every 12 weeks (q12w regimen). In certain embodiments, the present invention provides a method for treating nAMD in a patient, the method comprising: (a) administering to the patient two individual doses of a VEGF antagonist at 6-week interval (q6w regimen); and (b) assessing the patient for disease activity after the second dose of the VEGF antagonist, e.g., assessing the patient for disease activity between ≥0 and ≤6 weeks after the second dose of the VEGF antagonist; and, if presence of disease activity is identified after the second dose of the VEGF antagonist, administering to the patient a third dose of the VEGF antagonist 6 weeks after the administration of the second dose (q6w regimen), and (b) administering to the patient one or more additional doses of the VEGF antagonist, wherein each additional dose is administered in an administration interval of at least 8 weeks after the immediately preceding dose, e.g., once every 8 weeks (q8w regimen) to once every 12 weeks (q12w regimen), e.g., once every 8 weeks (q8w regimen) or once every 12 weeks (q12w regimen). In certain embodiments, the present invention provides a method for treating nAMD in a patient, the method comprising: (a) administering to the patient three individual doses of a VEGF antagonist at 6-week interval (q6w regimen); and (b) administering to the patient one or more additional doses of the VEGF antagonist, wherein each additional dose is administered in an administration interval of at least 8 weeks after the immediately preceding dose, e.g., once every 8 weeks (q8w regimen) to once every 12 weeks (q12w regimen), e.g., once every 8 weeks (q8w regimen) or once every 12 weeks (q12w regimen).

In a further aspect, the present invention provides a VEGF antagonist for use as a medicament for treating nAMD in a patient, wherein (a) the VEGF antagonist is administered to the patient as two individual doses at 6-week intervals (q6w regimen); (b) followed by one or more additional doses, wherein each additional dose is administered in an administration interval of at least 8 weeks after the immediately preceding dose, e.g., once every 8, 9, 10, 11, 12, 13, 14, 15, 16, 17, 18, 19, 20, 21, 22, 23, or 24 weeks, e.g., once every 8 weeks (q8w regimen) to once every 12 weeks (q12w regimen), e.g., once every 8 weeks (q8w regimen) or once every 12 weeks (q12w regimen). In certain embodiments, the present invention provides a VEGF antagonist for use as a medicament for treating nAMD in a patient, wherein (a) the VEGF antagonist is administered to the patient as two individual doses at 6-week intervals (q6w regimen); (b) followed by assessing the patient for disease activity after the second dose of the VEGF antagonist, e.g., assessing the patient for disease activity between ≥0 and ≤6 weeks after the second dose of the VEGF antagonist; and, if presence of disease activity is identified after the second dose of the VEGF antagonist, a third dose of the VEGF antagonist is administered to the patient 6 weeks after the administration of the second dose (q6w regimen); (c) followed by one or more additional doses, wherein each additional dose is administered in an administration interval of at least 8 weeks after the immediately preceding dose, e.g., once every 8 weeks (q8w regimen) to once every 12 weeks (q12w regimen), e.g., once every 8 weeks (q8w regimen) or once every 12 weeks (q12w regimen). In certain embodiments, the present invention provides a VEGF antagonist for use as a medicament for treating nAMD in a patient, wherein (a) the VEGF antagonist is administered to the patient as three individual doses at 6-week intervals (q6w regimen); (b) followed by one or more additional doses, wherein each additional dose is administered in an administration interval of at least 8 weeks after the immediately preceding dose, e.g., once every 8 weeks (q8w regimen) to once every 12 weeks (q12w regimen), e.g., once every 8 weeks (q8w regimen) or once every 12 weeks (q12w regimen).

In a further aspect, the present invention provides a pharmaceutical composition comprising a VEGF antagonist for use as a medicament for treating nAMD in a patient, wherein: (a) the pharmaceutical composition is administered to the patient as two individual doses at 6-week intervals (q6w regimen); (b) followed by one or more additional doses, wherein each additional dose is administered in an administration interval of at least 8 weeks after the immediately preceding dose, e.g., once every 8, 9, 10, 11, 12, 13, 14, 15, 16, 17, 18, 19, 20, 21, 22, 23, or 24 weeks, e.g., once every 8 weeks (q8w regimen) to once every 12 weeks (q12w regimen), e.g., once every 8 weeks (q8w regimen) or once every 12 weeks (q12w regimen). In certain embodiments, the present invention provides a pharmaceutical composition comprising a VEGF antagonist for use as a medicament for treating nAMD in a patient, wherein: (a) the pharmaceutical composition is administered to the patient as two individual doses at 6-week intervals (q6w regimen); (b) followed by assessing the patient for disease activity after the second dose of the pharmaceutical composition, e.g., assessing the patient for disease activity between ≥0 and ≤6 weeks after the second dose of the pharmaceutical composition; and, if presence of disease activity is identified after the second dose of the pharmaceutical composition, a third dose of the pharmaceutical composition is administered to the patient 6 weeks after the administration of the second dose (q6w regimen); (c) followed by one or more additional doses, wherein each additional dose is administered in an administration interval of at least 8 weeks after the immediately preceding dose, e.g., once every 8 weeks (q8w regimen) to once every 12 weeks (q12w regimen), e.g., once every 8 weeks (q8w regimen) or once every 12 weeks (q12w regimen). In certain embodiments, the present invention provides a pharmaceutical composition comprising a VEGF antagonist for use as a medicament for treating nAMD in a patient, wherein: (a) the pharmaceutical composition is administered to the patient as three individual doses at 6-week intervals (q6w regimen); (b) followed by one or more additional doses, wherein each additional dose is administered in an administration interval of at least 8 weeks after the immediately preceding dose, e.g., once every 8 weeks (q8w regimen) to once every 12 weeks (q12w regimen), e.g., once every 8 weeks (q8w regimen) or once every 12 weeks (q12w regimen).

In another aspect, the present invention provides use of a VEGF antagonist for the manufacture of a medicament for treating nAMD in a patient, the use comprising: (a) administering to the patient two individual doses of the VEGF antagonist at 6-week interval (q6w regimen); (b) administering to the patient one or more additional doses of the VEGF antagonist, wherein each additional dose is administered in an administration interval of at least 8 weeks after the immediately preceding dose, e.g., once every 8, 9, 10, 11, 12, 13, 14, 15, 16, 17, 18, 19, 20, 21, 22, 23, or 24 weeks, e.g., once every 8 weeks (q8w regimen) to once every 12 weeks (q12w regimen), e.g., once every 8 weeks (q8w regimen) or once every 12 weeks (q12w regimen). In certain embodiments, the present invention provides use of a VEGF antagonist for the manufacture of a medicament for treating nAMD in a patient, the use comprising: (a) administering to the patient two individual doses of the VEGF antagonist at 6-week interval (q6w regimen); (b) followed by assessing the patient for disease activity after the second dose of the VEGF antagonist, e.g., assessing the patient for disease activity between ≥0 and ≤6 weeks after the second dose of the VEGF antagonist; and, if presence of disease activity is identified after the second dose of the VEGF antagonist, administering a third dose of the VEGF antagonist 6 weeks after the administration of the second dose (q6w regimen); (c) administering to the patient one or more additional doses of the VEGF antagonist, wherein each additional dose is administered in an administration interval of at least 8 weeks after the immediately preceding dose, e.g., once every 8 weeks (q8w regimen) to once every 12 weeks (q12w regimen), e.g., once every 8 weeks (q8w regimen) or once every 12 weeks (q12w regimen). In certain embodiments, the present invention provides use of a VEGF antagonist for the manufacture of a medicament for treating nAMD in a patient, the use comprising: (a) administering to the patient three individual doses of the VEGF antagonist at 6-week interval (q6w regimen); (b) administering to the patient one or more additional doses of the VEGF antagonist, wherein each additional dose is administered in an administration interval of at least 8 weeks after the immediately preceding dose, e.g., once every 8 weeks (q8w regimen) to once every 12 weeks (q12w regimen), e.g., once every 8 weeks (q8w regimen) or once every 12 weeks (q12w regimen).

In certain embodiments, the methods and uses of the present invention do not comprise administering to the patient more than 3 doses in an administration interval of less than 8 week. In certain embodiments, the methods and uses of the present invention do not comprise administering to the patient more than 3 doses in an administration interval of 6 weeks.

In some embodiments, the methods and uses of the present disclosure comprise administering to a patient a VEGF antagonist as described herein, wherein the patient does not have (i) ocular inflammation, e.g., active ocular inflammation, and/or (ii) retinal vasculitis and/or retinal vascular occlusion, e.g., retinal vasculitis and/or retinal vascular occlusion in the presence of intraocular inflammation.

In certain embodiments, the methods and uses of the present invention comprise assessing the patient for disease activity after the second dose of the VEGF antagonist, e.g., assessing the patient for disease activity between ≥0 and ≤6 weeks after the second dose of the VEGF antagonist. In certain embodiments, if presence of disease activity is identified after the second dose of the VEGF antagonist, e.g., if presence of disease activity is identified between ≥0 and ≤6 weeks after the second dose of the VEGF antagonist, a third dose of the VEGF antagonist is administered to the patient 6 weeks after the second dose as part of the loading phase.

In some embodiments, according to the methods or the uses of the present disclosure, the first two or three q6w doses of the VEGF antagonist are followed by one or more doses of the VEGF antagonist in an administration interval as individualized by a physician based on a disease activity assessment and/or in an administration interval of at least 8 weeks, e.g., 8, 9, 10, 11, 12, 13, 14, 15, 16, 17, 18, 19, 20, 21, 22, 23, or 24 weeks. In some embodiments, according to the methods or the uses of the present disclosure, the first two or three q6w doses of the VEGF antagonist are followed by one or more doses of the VEGF antagonist in an administration interval as individualized by a physician based on a disease activity assessment and/or in an administration interval between ≥8 and ≤24 weeks, e.g., between ≥8 and ≤18 weeks (≥q8w to ≤q18w), between ≥8 and ≤12 weeks (≥q8w to ≤q12w). Suitably, the first two or three q6w doses of the VEGF antagonist are followed by administering to the patient one or more doses of the VEGF antagonist once every 8 weeks (q8w regimen) or once every 12 weeks (q12w regimen). In certain embodiments, the first two or three q6w doses of the VEGF antagonist are followed by one or more doses of the VEGF antagonist in an administration interval, e.g., an injection interval, of at least about two months, e.g., at least about three months, at least about four months, at least about five months, at least about six months. In a preferred embodiment, the first two or three q6w doses of the VEGF antagonist are followed by one or more doses of the VEGF antagonist in an administration interval, e.g., an injection interval, of at least about two months. In a more preferred embodiment, the first two or three q6w doses of the VEGF antagonist are followed by one or more doses of the VEGF antagonist in an administration interval, e.g., an injection interval, of at least about three months.

In certain embodiments, a Disease Activity Assessment (DAA) is conducted at all scheduled treatment visits. In some embodiments, the methods or the uses of the present disclosure comprise assessing the patient for disease activity before or after administering a dose of the VEGF antagonist. In some embodiments, the methods or the uses of the present disclosure comprise assessing the patient for disease activity before or after administering every q6w or q8w or q12w dose of the VEGF antagonist. The assessment can determine if a patient stays on the current interval or switches to a different interval. In certain embodiments, the methods and uses of the present invention comprise assessing the patient for disease activity after administering the second q6w dose of the VEGF antagonist or/and before or after administering every q8w or q12w dose of the VEGF antagonist. In certain embodiments, if presence of disease activity is identified after the second q6w dose of the VEGF antagonist, a third dose of the VEGF antagonist is administered 6 weeks after the administration of the second dose. In certain embodiments, if presence of disease activity is identified after a q12w dose of the VEGF antagonist, the patient is switched to a q8w regimen of the VEGF antagonist. In certain embodiments, if no disease activity is identified after a q8w dose of the VEGF antagonist, the patient is switched to a q12w regimen of the VEGF antagonist.

Suitably, the disease activity may be assessed based on visual function, retinal structure and leakage. An assessment as described herein preferably includes one or more of the following tests to assess activity of a VEGF antagonist (e.g., brolucizumab) on visual function, retinal structure and leakage: (i) best corrected visual acuity (BCVA), (ii) visual acuity (VA), (iii) central subfield thickness (CSFT), (iv) presence of intraretinal cysts/fluid, (v) ETDRS DRSS score based on 7-field stereo Color Fundus Photography (CFP), (vi) anatomical retinal evaluation by Optical Coherence Tomography (OCT), standard or wide-field Fluorescein Angiography (FA), OCT angiography, and/or wide-field CFP/FA, (vii) peripheral visual field assessed by perimetry, (viii) contrast sensitivity.

Visual acuity can be assessed using best correction determined from protocol refraction (BCVA). BCVA measurements can be taken, for example, in a sitting position using ETDRS-like visual acuity testing charts.

Optical Coherence Tomography (OCT), color fundus photography and fluorescein angiography can be assessed according to methods known to those of skill in the art.

The CST is the average thickness of circular 1 mm area centered around the fovea measured from retinal pigment epithelium (RPE) to the internal limiting membrane (ILM), inclusively. CST can be measured, for example, using spectral domain Optical Coherence Tomography (SD-OCT).

Means of performing the above tests are well understood and commonly used by those skilled in the art.

Suitably, the disease activity may be assessed based on one or more of the following: (i) best corrected visual acuity (BCVA), (ii) visual acuity (VA), (iii) central subfield thickness (CSFT), and (iv) presence of intraretinal cysts/fluid. The presence of disease activity includes one or more of the following: (i) decrease in Best Corrected Visual Acuity (BCVA), (ii) decrease in Visual Acuity (VA), (iii) increase or lack of reduction in Central Subfield Thickness (CSFT), (iv) new or persistent or recurrent Intraretinal Cysts (IRC) and/or Intraretinal Fluid (IRF) and/or Subretinal Fluid (SRF). Fluid measured in the eye can be intraretinal and/or subretinal fluid.

In one embodiment, assessments of disease activity to establish patient's disease status occurs at baseline (e.g., Week 0; first treatment with a VEGF antagonist; prior to the last administration of a VEGF antagonist). The assessment of the disease activity (DAA) during treatment regimens is at the discretion of the person making the assessment (e.g., the treatment provider), and is based on changes in vision and anatomical and morphological and clinical parameters with reference to patients' baseline disease status (e.g., at Week 0; first treatment with a VEGF antagonist; prior to the last administration of a VEGF antagonist).

In specific embodiments, the presence of disease activity includes one or more of the following:
(i) decrease in BCVA of ≥2 letters, e.g., decrease in BCVA of ≥3 letters, decrease in BCVA of ≥4 letters, in particular decrease in BCVA of ≥5 letters, more particularly wherein the decrease in BCVA is observed after the last administration of the VEGF antagonist (e.g., brolucizumab) compared to a baseline BCVA, wherein the baseline BCVA was assessed prior to the last administration of the VEGF antagonist;
(ii) decrease in VA of ≥1 letters, e.g., decrease in VA of ≥2 letters, in particular decrease in VA of ≥3 letters, more particularly wherein the decrease in VA is observed after the last administration of the VEGF antagonist (e.g., brolucizumab) compared to a baseline VA, wherein the baseline VA was assessed prior to the last administration of the VEGF antagonist;
(iii) CSFT increase ≥25 μm, e.g., CSFT increase ≥50 μm, in particular CSFT increase ≥75 μm, more particularly wherein the CSFT increase is observed after the last administration of the VEGF antagonist (e.g., brolucizumab) compared to a baseline CSFT, wherein the baseline CSFT was assessed prior to the last administration of the VEGF antagonist;
(iv) new or persistent or recurrent intraretinal cysts (IRC) and/or intraretinal fluid (IRF) and/or subretinal fluid (SRF), in particular wherein new or persistent or recurrent intraretinal cysts (IRC) and/or intraretinal fluid (IRF) and/or subretinal fluid (SRF) are observed at after the last administration of the VEGF antagonist (e.g., brolucizumab) compared to a baseline RC and/or IRF and/or SRF, wherein the baseline RC and/or IRF and/or SRF was assessed prior to the last administration of the VEGF antagonist.

Where disease activity is present (for example, loss of letters measured by BCVA, increase in CST, increased fluid accumulation, and or increased severity of disease compared with baseline reading for a patient or compared with any previous assessment), a more frequent dosing interval is prescribed going forward. Where improvement of disease activity is observed, a less frequent dosing interval is prescribed.

In certain embodiments, the dosing frequency is adjusted based on the outcome of disease activity assessments, for example using pre-defined visual and anatomic criteria. In one embodiment, dosing frequency of a VEGF antagonist (e.g., brolucizumab) can be adjusted by decreasing the dosing interval from once every 24 weeks (q24w) to once every 18 weeks (q18w). In one embodiment, dosing frequency of a VEGF antagonist (e.g., brolucizumab) can be adjusted by decreasing the dosing interval from once every 18 weeks (q18w) to once every 12 weeks (q12w). In one embodiment, dosing frequency of a VEGF antagonist (e.g., brolucizumab) can be adjusted by decreasing the dosing interval from once every 12 weeks (q12w) to once every 8 weeks (q8w) based on the disease activity assessment at any scheduled treatment visit. In another embodiment, dosing frequency of a VEGF antagonist (e.g., brolucizumab) can be adjusted by increasing the dosing interval from once every 8 weeks (q8w) to once every 12 weeks (q12w) based on the disease activity assessment at any scheduled treatment visit. In another embodiment, dosing frequency of a VEGF antagonist (e.g., brolucizumab) can be adjusted by increasing the dosing interval from once every 12 weeks (q12w) or to once every 18 weeks (q18w) or to once every 24 weeks (q24w) based on the disease activity assessment at any scheduled treatment visit. When disease activity is identified as described herein, the treatment regimen can be changed, e.g., from every 12 weeks to every 8 weeks (i.e., q8w). In some cases, a patient might be on a 12-week interval regimen for some time, and then switch to an 8-week interval, and then switch back to the 12-week interval. Thus, patients may not stay on one interval regimen, and may go back and forth depending on assessments according to the criteria set forth herein.

### Anti-VEGF Antagonists

In one embodiment, the VEGF antagonist of the disclosure is any licensed anti-VEGF drug such as brolucizumab, ranibizumab or aflibercept. In one embodiment, the VEGF antagonist of the disclosure is an anti-VEGF antibody (such as brolucizumab or ranibizumab or bevacizumab or a bi-specific antibody such as faricimab) or an anti-VEGF DARPin (such as abicipar) or a soluble VEGF receptor (e.g., a fusion protein composed of the VEGF receptor domains, such as a fusion protein composed of the combination between VEGF receptor domains with the Fc fragment of human immunoglobulin with the Fc fragment of human immunoglobulin, e.g., conbercept, aflibercept) or AAV containing a sequence encoding for an anti-VEGF antibody (such as RGX-314 from Regenxbio), or AAV containing a sequence encoding the VEGF receptor domains, e.g., conbercept (such as ADVM-022 from Adverum) or any licensed anti-VEGF drug (such as brolucizumab, ranibizumab or aflibercept).

In certain embodiments, the VEGF antagonist of the disclosure is an anti-VEGF antibody, e.g., a single chain antibody (scFv) or Fab fragment.

In certain embodiments, the VEGF antagonist of the disclosure is an anti-VEGF antibody, e.g., anti-VEGF antibodies described in WO 2009/155724, the entire contents of which are hereby incorporated by reference.

In one embodiment, the VEGF antagonist of the disclosure is an anti-VEGF antibody comprising a variable heavy chain having the sequence as set forth in SEQ ID NO: 1 and a variable light chain having the sequence as set forth in SEQ ID NO: 2.

VH:
SEQ ID NO. 1
EVQLVESGGGLVQPGGSLRLSCTASGFSLTDYYYMTWVRQAPGKGLEWV
GFIDPDDDPYYATWAKGRFTISRDNSKNTLYLQMNSLRAEDTAVYYCAG
GDHNSGWGLDIWGQGTLVTVSS
VL:
SEQ ID NO. 2
EIVMTQSPSTLSASVGDRVIITCQASEIIHSWLAWYQQKPGKAPKLLIY
LASTLASGVPSRFSGSGSGAEFTLTISSLQPDDFATYYCQNVYLASTNG
ANFGQGTKLTVLG

In another embodiment, the VEGF antagonist of the disclosure is an anti-VEGF antibody comprising the sequence as set forth in SEQ ID NO: 3.

EIVMTQSPSTLSASVGDRVIITCQASEIIHSWLAWYQQKPGKAPKLLIY
LASTLASGVPSRFSGSGSGAEFTLTISSLQPDDFATYYCQNVYLASTNG
ANFGQGTKLTVLGGGGGSGGGGSGGGGSGGGGSEVQLVESGGGLVQPGG
SLRLSCTASGFSLTDYYYMTWVRQAPGKGLEWVGFIDPDDDPYYATWAK
GRFTISRDNSKNTLYLQMNSLRAEDTAVYYCAGGDHNSGWGLDIWGQGT
LVTVSS

In another embodiment, the VEGF antagonist of the disclosure is an anti-VEGF antibody comprising the sequence as set forth in SEQ ID NO: 4 (brolucizumab). The sequence of brolucizumab is set forth in SEQ ID NO: 4 and comprises the sequence of SEQ ID NO: 3. A methionine derived from the start codon in an expression vector is present in the final protein in cases where it has not been cleaved posttranslationally as follows.

(SEQ ID NO: 4)
MEIVMTQSPS TLSASVGDRV IITCQASEII
HSWLAWYQQK PGKAPKLLIY LASTLASGVP
SRFSGSGSGA EFTLTISSLQ PDDFATYYCQ
NVYLASTNGA NFGQGTKLTV LGGGGGSGGG
GSGGGGSGGG GSEVQLVESG GGLVQPGGSL
RLSCTASGFS LTDYYYMTWV RQAPGKGLEW
VGFIDPDDDP YYATWAKGRF TISRDNSKNT
LYLQMNSLRA EDTAVYYCAG GDHNSGWGLD
IWGQGTLVTV SS

In another embodiment, the VEGF antagonist of the disclosure is an anti-VEGF antibody comprising three light chain CDRs (CDRL1, CDRL2, and CDRL3) and three heavy chain CDRs (CDRH1, a CDRH2, a CDRH3) as follows:

CDRL1
SEQ ID NO: 5
QASEIIHSWLA
CDRL2
SEQ ID NO: 6
LASTLAS
CDRL3
SEQ ID NO: 7
QNVYLASTNGAN
CDRH1
SEQ ID NO: 8
GFSLTDYYYMT
CDRH2
SEQ ID NO: 9
FIDPDDDPYYATWAKG
CDRH3
SEQ ID NO: 10
GDHNSGWGLDI

Brolucizumab, is a humanized single-chain Fv (scFv) antibody fragment inhibitor of VEGF with a molecular weight of ˜26 kDa. It is an inhibitor of VEGF-A and works by binding to the receptor binding site of the VEGF-A molecule, thereby preventing the interaction of VEGF-A with its receptors VEGFR1 and VEGFR2 on the surface of endothelial cells. Increased levels of signaling through the VEGF pathway are associated with pathologic ocular angiogenesis and retinal edema. Inhibition of the VEGF pathway has been shown to inhibit the growth of neovascular lesions and resolve retinal edema in patients with nAMD.

In certain embodiments, the VEGF antagonist of the disclosure is administered by an injection. In certain embodiments, the VEGF antagonist of the disclosure is administered by an intravitreal injection.

In some embodiments, the VEGF antagonist of the disclosure is brolucizumab and is administered at a dose of about 1, about 2, about 3, about 4, about 5, or about 6 mg (e.g., about 6 mg/0.05 mL) as an intravitreal injection. In certain embodiments, the VEGF antagonist of the disclosure is brolucizumab and is administered at a dose of 1, 2, 3, 4, 5, or 6 mg (e.g., 6 mg/0.05 mL) as an intravitreal injection.

### Pharmaceutical Preparations

In one aspect, the methods or uses of the disclosure comprise the use of pharmaceutical formulations or pharmaceutical compositions comprising a VEGF antagonist, e.g., an anti-VEGF antibody. The term “pharmaceutical formulation” or “pharmaceutical composition” refers to preparations which are in such form as to permit the biological activity of the antagonist, e.g., antibody or antibody derivative, to be unequivocally effective, and which contain no additional components which are toxic to the subjects to which the formulation or composition would be administered. “Pharmaceutically acceptable” excipients (vehicles, additives) are those which can reasonably be administered to a subject mammal to provide an effective dose of the active ingredient employed.

A “stable” formulation is one in which a therapeutic agent, e.g. a VEGF antagonist, e.g., an anti-VEGF antibody or antibody derivative thereof, essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N. Y., Pubs. (1991) and Jones, A. Adv. Drug Delivery Rev. 10:29-90 (1993), for example. Stability can be measured at a selected temperature for a selected time period. Preferably, the formulation is stable at room temperature (about 30° C.) or at 40° C. for at least 1 week and/or stable at about 2-8° C. for at least 3 months to 2 years. Furthermore, the formulation is preferably stable following freezing (to, e.g., −70° C.) and thawing of the formulation.

An antagonist, e.g., an antibody or antibody derivative, “retains its physical stability” in a pharmaceutical formulation if it meets the defined release specifications for aggregation, degradation, precipitation and/or denaturation upon visual examination of color and/or clarity, or as measured by UV light scattering or by size exclusion chromatography, or other suitable art recognized methods.

An antagonist, e.g., an antibody or antibody derivative, “retains its chemical stability” in a pharmaceutical formulation, if the chemical stability at a given time is such that the compound, e.g., protein, is considered to still retain its biological activity as defined below. Chemical stability can be assessed by detecting and quantifying chemically altered forms of the protein. Chemical alteration may involve size modification (e.g. clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g. occurring as a result of deamidation) which can be evaluated by ion-exchange chromatography, for example.

An antagonist, e.g., an antibody or antibody derivative, “retains its biological activity” in a pharmaceutical formulation, if the biological activity of the antibody at a given time is within about 10% (within the errors of the assay) of the biological activity exhibited at the time the pharmaceutical formulation was prepared as determined in an antigen binding assay, for example. Other “biological activity” assays for antibodies are elaborated herein below.

By “isotonic” is meant that the formulation of interest has essentially the same osmotic pressure as human blood. Isotonic formulations will generally have an osmotic pressure from about 250 to 350 mOsm. Isotonicity can be measured using a vapor pressure or ice-freezing type osmometer, for example.

A “polyol” is a substance with multiple hydroxyl groups, and includes sugars (reducing and non-reducing sugars), sugar alcohols and sugar acids. Preferred polyols herein have a molecular weight which is less than about 600 kD (e.g. in the range from about 120 to about 400 kD). A “reducing sugar” is one which contains a hemiacetal group that can reduce metal ions or react covalently with lysine and other amino groups in proteins and a “non-reducing sugar” is one which does not have these properties of a reducing sugar. Examples of reducing sugars are fructose, mannose, maltose, lactose, arabinose, xylose, ribose, rhamnose, galactose and glucose. Non-reducing sugars include sucrose, trehalose, sorbose, melezitose and raffinose. Mannitol, xylitol, erythritol, threitol, sorbitol and glycerol are examples of sugar alcohols. As to sugar acids, these include L-gluconate and metallic salts thereof. Where it is desired that the formulation is freeze-thaw stable, the polyol is preferably one which does not crystallize at freezing temperatures (e.g. −20° C.) such that it destabilizes the antibody in the formulation. Non-reducing sugars such as sucrose and trehalose are the preferred polyols herein, with trehalose being preferred over sucrose, because of the superior solution stability of trehalose.

As used herein, “buffer” refers to a buffered solution that resists changes in pH by the action of its acid-base conjugate components. The buffer of this disclosure has a pH in the range from about 4.5 to about 8.0; preferably from about 5.5 to about 7. Examples of buffers that will control the pH in this range include acetate (e.g. sodium acetate), succinate (such as sodium succinate), gluconate, histidine, citrate and other organic acid buffers. Where a freeze-thaw stable formulation is desired, the buffer is preferably not phosphate.

In a pharmacological sense, in the context of the present disclosure, a “therapeutically effective amount” of a therapeutic agent, e.g., a VEGF antagonist, e.g., an anti-VEGF antibody or antibody derivative, refers to an amount effective in the prevention or treatment of a disorder for the treatment of which the antagonist, e.g., antibody or antibody derivative, is effective. This includes chronic and acute disorders or diseases including those pathological conditions which predispose the mammal to the disorder in question.

A “preservative” is a compound which can be included in the formulation to essentially reduce bacterial action therein, thus facilitating the production of a multi-use formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The most preferred preservative herein is benzyl alcohol.

The pharmaceutical compositions used in present disclosure comprise a VEGF antagonist, preferably an anti-VEGF antibody (e.g., an anti-VEGF antibody comprising the variable light chain sequence of SEQ ID NO: 1 and the variable heavy chain sequence of SEQ ID NO: 2, such as brolucizumab), together with at least one physiologically acceptable carrier or excipient. Pharmaceutical compositions may comprise, for example, one or more of water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. As noted above, other active ingredients may (but need not) be included in the pharmaceutical compositions provided herein.

A carrier is a substance that may be associated with an antibody or antibody derivative prior to administration to a patient, often for the purpose of controlling stability or bioavailability of the compound. Carriers for use within such formulations are generally biocompatible, and may also be biodegradable. Carriers include, for example, monovalent or multivalent molecules such as serum albumin (e.g., human or bovine), egg albumin, peptides, polylysine and polysaccharides such as aminodextran and polyamidoamines. Carriers also include solid support materials such as beads and microparticles comprising, for example, polylactate polyglycolate, poly (lactide-co-glycolide), polyacrylate, latex, starch, cellulose or dextran. A carrier may bear the compounds in a variety of ways, including covalent bonding (either directly or via a linker group), noncovalent interaction or admixture.

Pharmaceutical compositions may be formulated for any appropriate manner of administration, including, for example, topical, intraocular, oral, nasal, rectal or parenteral administration. In certain embodiments, compositions in a form suitable for intraocular injection, such as intravitreal injection, are preferred.

The pharmaceutical composition may be prepared as a sterile injectible aqueous or oleaginous suspension in which the active agent (i.e. VEGF antagonist), depending on the vehicle and concentration used, is either suspended or dissolved in the vehicle. Such a composition may be formulated according to the known art using suitable dispersing, wetting agents and/or suspending agents such as those mentioned above. Among the acceptable vehicles and solvents that may be employed are water, 1,3-butanediol, Ringer's solution and isotonic sodium chloride solution. In addition, sterile, fixed oils may be employed as a solvent or suspending medium. For this purpose any bland fixed oil may be employed, including synthetic mono- or diglycerides. In addition, fatty acids such as oleic acid may be used in the preparation of injectible compositions, and adjuvants such as local anesthetics, preservatives and/or buffering agents can be dissolved in the vehicle.

An aqueous formulation of a VEGF antagonist, e.g., an anti-VEGF antibody (e.g., brolucizumab), used in the methods or uses of the disclosure is prepared in a pH-buffered solution. Preferably, the buffer of such aqueous formulation has a pH in the range from about 4.5 to about 8.0, preferably from about 5.5 to about 7.0, most preferably about 6.75. In one embodiment, the pH of an aqueous pharmaceutical composition of the disclosure is about 7.0-7.5, or about 7.0-7.4, about 7.0-7.3, about 7.0-7.2, about 7.1-7.6, about 7.2-7.6, about 7.3-7.6 or about 7.4-7.6. In one embodiment, an aqueous pharmaceutical composition of the disclosure has a pH of about 7.0, about 7.1, about 7.2, about 7.3, about 7.4, about 7.5 or about 7.6. In a preferred embodiment, the aqueous pharmaceutical composition has a pH of ≥7.0 In a preferred embodiment, the aqueous pharmaceutical composition has a pH of about 7.2. Examples of buffers that will control the pH within this range include acetate (e.g. sodium acetate), succinate (such as sodium succinate), gluconate, histidine, citrate (e.g. sodium citrate) and other organic acid buffers. The buffer concentration can be from about 1 mM to about 50 mM, preferably from about 5 mM to about 30 mM, depending, for example, on the buffer and the desired isotonicity of the formulation. In a preferred embodiment, the aqueous pharmaceutical composition comprises 15 mM sodium citrate buffer.

A polyol, which acts as a tonicifier, may be used to stabilize an antibody in an aqueous formulation. In preferred embodiments, the polyol is a non-reducing sugar, such as sucrose or trehalose, preferably sucrose. If desired, the polyol is added to the formulation in an amount that may vary with respect to the desired isotonicity of the formulation. Preferably the aqueous formulation is isotonic, in which case suitable concentrations of the polyol in the formulation are in the range from about 1% to about 15% w/v, preferably in the range from about 2% to about 10% w/v, for example. However, hypertonic or hypotonic formulations may also be suitable. The amount of polyol added may also alter with respect to the molecular weight of the polyol. For example, a lower amount of a monosaccharide (e.g. mannitol) may be added, compared to a disaccharide (such as trehalose).

A surfactant is also added to an aqueous antibody formulation. Exemplary surfactants include nonionic surfactants such as polysorbates (e.g. polysorbates 20, 80 etc) or poloxamers (e.g. poloxamer 188). The amount of surfactant added is such that it reduces aggregation of the formulated antibody/antibody derivative and/or minimizes the formation of particulates in the formulation and/or reduces adsorption. For example, the surfactant may be present in the formulation in an amount from about 0.001% to about 0.5%, preferably from about 0.005% to about 0.2%, preferably from about 0.01% to about 0.02%, and most preferably about 0.02%.

In one embodiment, an aqueous antibody formulation used in the methods or uses of the disclosure is essentially free of one or more preservatives, such as benzyl alcohol, phenol, m-cresol, chlorobutanol and benzethonium Cl. In another embodiment, a preservative may be included in the formulation, particularly where the formulation is a multidose formulation. The concentration of preservative may be in the range from about 0.1% to about 2%, most preferably from about 0.5% to about 1%. One or more other pharmaceutically acceptable carriers, excipients or stabilizers such as those described in Remington's Pharmaceutical Sciences 21st edition, Osol, A. Ed. (2006) may be included in the formulation provided that they do not adversely affect the desired characteristics of the formulation. Acceptable carriers, excipients or stabilizers are non-toxic to recipients at the dosages and concentrations employed and include: additional buffering agents, co-solvents, antioxidants including ascorbic acid and methionine, chelating agents such as EDTA, metal complexes (e.g. Zn-protein complexes), biodegradable polymers such as polyesters, and/or salt-forming counterions such as sodium.

Formulations to be used for in vivo administration must be sterile. This is readily accomplished by filtration through sterile filtration membranes, prior to, or following, preparation of the formulation.

In one embodiment, the VEGF antagonist of the disclosure is administered to an eye of a subject in need of treatment in accordance with known methods for ocular delivery. Preferably, the subject is a human, the VEGF antagonist A is an anti-VEGF antibody (preferably brolucizumab), and the antibody is administered directly to an eye. Administration to a patient can be accomplished, for example, by intravitreal injection.

The VEGF antagonist in the methods and uses of the disclosure can be administered as the sole treatment or in conjunction with other drugs or therapies useful in treating the condition in question.

A preferred formulation for brolucizumab for intravitreal injection comprises about 4.5% to 11% (w/v) sucrose, 5-20 mM sodium citrate, and 0.001% to 0.05% (w/v) polysorbate 80, wherein the pH of the formulation is about 7.0 to about 7.4. One such formulation comprises 5.9% (w/v) sucrose, 10 mM sodium citrate, 0.02% (w/v) polysorbate 80, pH of 7.2, and 6 mg of brolucizumab. Another such formulation comprises 6.4% (w/v) or 5.8% sucrose, 12 mM or 10 mM sodium citrate, 0.02% (w/v) polysorbate 80, pH of 7.2, and 3 mg of brolucizumab. One such formulation comprises 6.75% (w/v) sucrose, 15 mM sodium citrate, 0.02% (w/v) polysorbate 80, pH of 7.2, and 6 mg of brolucizumab. Preferred concentrations of brolucizumab are about 120 mg/ml and about 60 mg/ml. Doses can be delivered, for example as 6 mg/50 μL and 3 mg/50 μL concentrations.

### Dosage

A dose used in the methods or uses of the disclosure is based on the specific disease or condition being treated, and is a therapeutically effective dose. Amounts effective for this use will depend upon the severity of the disorder being treated and the general state of the patient's own immune system. The dose amount can be readily determined using known dosage adjustment techniques by a physician having ordinary skill in treatment of the disease or condition. The therapeutically effective amount of a VEGF antagonist used in the methods or uses of the disclosure is determined by taking into account the desired dose volumes and mode(s) of administration, for example. Typically, therapeutically effective compositions are administered in a dosage ranging from 0.001 mg/ml to about 200 mg/ml per dose.

In one embodiment of the present disclosure, the VEGF antagonist used in the methods or uses of the disclosure is brolucizumab, and a dosage thereof used in the methods or uses of the disclosure is about 60 mg/ml to about 120 mg/ml (for example, a dosage is 60, 70, 80, 90, 100, 110, or 120 mg/ml). In a preferred embodiment, the dosage of the VEGF antagonist used in the methods or uses of the disclosure is 60 mg/ml or 120 mg/ml. Suitably, each dose is 50 μL, e.g., each dose is 6 mg/50 μL os 3 mg/50 μL.

In certain embodiments, a dose of the VEGF antagonist is administered directly to an eye of a patient. In one embodiment, a dose of the VEGF antagonist per eye is at least about 0.5 mg up to about 6 mg. Preferred doses per eye include about 0.5 mg, 0.6 mg, 0.7 mg, 0.8 mg, 0.9 mg, 1.0 mg, 1.2 mg, 1.4 mg, 1.6 mg, 1.8 mg, 2.0 mg, 2.5 mg, 3.0 mg, 3.5 mg, 4.0 mg, 4.5 mg, 5.0 mg, 5.5 mg, and 6.0 mg. In one embodiment, a dose per eye is at least about 3 mg up to about 6 mg, in particular about 3 mg or about 6 mg. Doses can be administered in various volumes suitable for ophthalmic administration, such as 50 μl or 100 μl, for example, including 3 mg/50 μl or 6 mg/50 μl. Smaller volumes can also be used, including 20 μl or less, for example about 20 μl, about 10 μl, or about 8.0 μl. In certain embodiments, a dose of 2.4 mg/20 μl, 1.2 mg/10 μl or 1 mg/8.0 μl (e.g., 1 mg/8.3 μl) is delivered to an eye of a patient for treating or ameliorating one or more of the diseases and disorders described above. Delivery can be, for example, by an injections, e.g., an intravitreal injection.

In specific embodiments, the VEGF antagonist of the disclosure is brolucizumab and is administered at a dose of about 1, about 2, about 3, about 4, about 5, or about 6 mg (e.g., about 6 mg/0.05 mL), e.g., 1, 2, 3, 4, 5, or 6 mg (e.g., 6 mg/0.05 mL), as an injections, e.g., an intravitreal injection.

### Kits

The disclosure also provides a kit, comprising: a drug container (e.g., a vial or a pre-filled syringe) comprising a VEGF antagonist (e.g., brolucizumab), and instructions for using the VEGF antagonist for treating a patient diagnosed with nAMD.

In one embodiment, the instructions indicate that a VEGF antagonist (e.g., brolucizumab) is to be administered to a patient as two individual doses at 6-week intervals (q6w regimen); followed by one or more additional doses, wherein each additional dose is to be administered in an administration interval of at least 8 weeks after the immediately preceding dose, e.g., 8, 9, 10, 11, 12, 13, 14, 15, 16, 17, 18, 19, 20, 21, 22, 23, or 24 weeks, e.g., once every 8 weeks (q8w regimen) to once every 12 weeks (q12w regimen), e.g., once every 8 weeks (q8w regimen) or once every 12 weeks (q12w regimen).

In a more specific embodiment, the instructions indicate that a VEGF antagonist (e.g., brolucizumab) is to be administered to a patient as two individual doses at 6-week intervals (q6w regimen); optionally, followed by assessing the patient for disease activity after the second dose of the VEGF antagonist, e.g., assessing the patient for disease activity between ≥0 and ≤6 weeks after the second dose of the VEGF antagonist; and, if presence of disease activity is identified after the second dose of the VEGF antagonist, a third dose of the VEGF antagonist is to be administered to the patient 6 weeks after the administration of the second dose (q6w regimen); followed by one or more additional doses, wherein each additional dose is to be administered in an administration interval of at least 8 weeks after the immediately preceding dose, e.g., 8, 9, 10, 11, 12, 13, 14, 15, 16, 17, 18, 19, 20, 21, 22, 23, or 24 weeks, e.g., once every 8 weeks (q8w regimen) to once every 12 weeks (q12w regimen), e.g., once every 8 weeks (q8w regimen) or once every 12 weeks (q12w regimen).

In a more specific embodiment, the instructions indicate that the VEGF antagonist (e.g., brolucizumab) is to be administered to a patient at a dose of 3 mg or 6 mg, preferably 6 mg, more preferably 6 mg/50 μL.

The disclosure also provides a kit, comprising: a drug container (e.g., a vial or a pre-filled syringe) comprising the VEGF antagonist (e.g., brolucizumab), and instructions for using the VEGF antagonist for treating a patient diagnosed with nAMD.

In one embodiment, the kit comprises one or more 3 mg or 6 mg doses of brolucizumab, each dose provided in a single use container, e.g., vial, containing sufficient brolucizumab to deliver a 3 mg or 6 mg, preferably 6 mg, dose when administering a volume of 0.05 mL or in a prefilled syringe containing 3 mg or 6 mg, e.g., 3 mg/50 μL or 6 mg/50 μL, preferably 6 mg, e.g., 6 mg/50 μL, of brolucizumab.

In one embodiment, the instructions further indicate that a treatment provider (e.g., a physician or other qualified medical professional) can adjust the dosing interval from once every 12 weeks to once every 8 weeks if disease activity is observed in the treated eye.

In another embodiment, the instructions further indicate that a treatment provider (e.g., a physician or other qualified medical professional) can extend the dosing interval from once every 8 weeks to once every 12weeks, if no disease activity is observed in the treated eye.

In yet another embodiment, the instructions further indicate that a VEGF antagonist is administered on an as needed basis, i.e., pro re nata (PRN), at the discretion of a treatment provider (e.g., a physician or other qualified medical professional) based on visual and/or anatomical outcomes to determine disease activity during the maintenance phase.

The various features, aspects and embodiments of the present invention, referred to in individual sections above apply, as appropriate, to other sections, mutatis mutandis. Consequently features specified in one section may be combined with features specified in other sections, as appropriate.

Those skilled in the art will recognize, or be able to ascertain using no more than routine experimentation, many equivalents to the specific aspects and embodiments of the invention described herein. Such equivalents are intended to be encompassed by the following claims.

All references cited herein, including patents, patent applications, papers, publications, text books, and the like, and the references cited therein, to the extent that they are not already, are hereby incorporated herein by reference in their entirety

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1: Posology Simulations of Predicted Human Intraocular Drug PK and VEGF Inhibition

### Background and Objective

Brolucizumab is a humanized, single-chain variable fragment (scFv) antibody developed for the treatment of wet age-related macular degeneration (wet AMD). Like other anti-VEGF biologics approved for use in wet AMD, brolucizumab is locally administered via an intravitreal injection. The approved dosing regimen for brolucizumab consists of a loading phase (3 monthly injections of 6 mg) followed by a maintenance phase (injections every 8 or 12 weeks; Q8W or Q12W).

The objective of the modeling activity was to predict retinal VEGF inhibition for alternative dosing regimens, including changes to the loading and/or maintenance phases. A quantitative systems pharmacology (QSP) modeling approach was used to predict the human ocular pharmacokinetics (PK) and VEGF inhibition for brolucizumab for alternative dosing regimens. The model was adapted from a series of models that link IVT administration of anti-VEGF biologics to intraocular PKPD and clearance from the eye (Hutton-Smith L A, et al (2016) Molecular Pharmaceutics; 13 (9): 2941-2950; Hutton-Smith L A, et al (2017) Molecular Pharmaceutics; 14 (8): 2690-2696; Hutton-Smith L A, et al (2018) Molecular Pharmaceutics; 15 (7): 2770-2784; Caruso A, et al (2020) Molecular Pharmaceutics; 17 (2): 695-709). The application of the model to brolucizumab considered the rapid distribution of this scFv antibody in the eye due to its small size (hydrodynamic radius) and formation of inhibited complexes with VEGF. The simulated clearance of free and VEGF-bound brolucizumab from the eye (i.e., the ocular half-life of 4.4 days) is consistent with the absorption rate identified in the clinical pharmacology analysis of serum concentrations of brolucizumab in patients. The analysis considered intra-patient variability in ocular VEGF levels by repeating simulations with varied retinal VEGF synthesis rates.

Simulations were performed to compare an approved brolucizumab dosing regimen (a 3× Q4W loading dose period followed by Q8W or Q12W maintenance doses) with an alternative regimen consisting of a 2× Q6W loading dose period followed by Q8W or Q12W maintenance doses.

### Materials and Methods

### Model Structure

A three compartment PKPD model below that describes distribution of drug within the eye following IVT administration (pharmacokinetics; PK) and the dynamics of VEGF production and inhibition by drug (pharmacodynamics; PD) was constructed using the MATLAB SimBiology software (MathWorks®, Natick, MA USA):

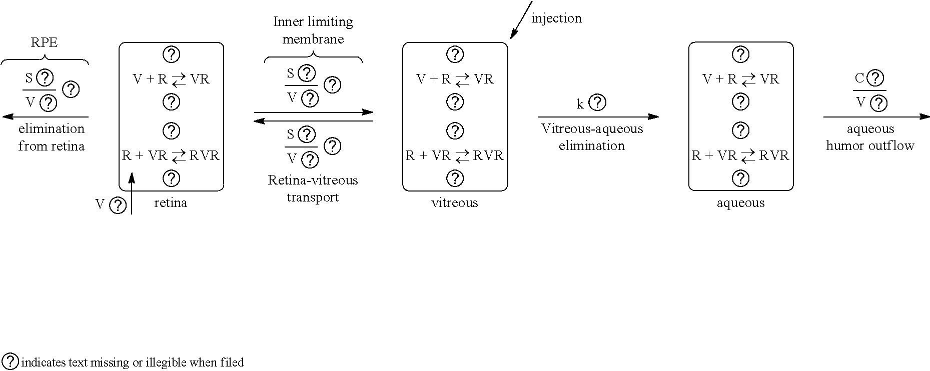

Simulations were performed according to manufacturer's instructions and using computational modeling techniques which are common in the art. The PKPD model was adapted from a published model in the literature (Hutton-Smith L A, et al (2018) Molecular Pharmaceutics; 15 (7): 2770-2784) that describes PKPD within the human eye for ranibizumab. The adapted model allows for simulations in which a VEGF antagonist (e.g., brolucizumab) is administered at specified doses and times. VEGF is present in the eye in a dimeric form consisting of two VEGF monomers and can thus form complexes with one VEGF antagonist or two VEGF antagonists.

The model contains 3 compartments (Table 1).

TABLE 1
Model Compartments.

| Name | Value | Units | Description |
|---|---|---|---|
| Ret | 0.22 | mL | Retina |
| Vit | 4.5 | mL | Vitreous |
| Aq | 0.16 | mL | Aqueous |

Following IVT administration of drug into the vitreous compartment, drug distributes between the retina, vitreous, and aqueous compartments and irreversibly distributes out of the eye via the retina and aqueous compartments. The term “ocular half-life” can generally refer to the summed rates at which drug distributes out of the aqueous and retina compartments of the eye (i.e. clears from the eye). VEGF is generally understood to distribute into the eye via the retina and is described within the model as a zero-order reaction in which new VEGF appears in the retina (i.e. “VEGF synthesis” or “VEGF production”). Distribution of VEGF within and out of the eye follows the same formalism as for the VEGF antagonists.

Species shown in the diagram in the retina, the vitreous and the aqueous compartments are presented in Tables 2, 3 and 4, respectively. Values of the parameters presented in the diagram are given in Table 5.

TABLE 2
Table of Species (state variables) in the Retina (Ret) Compartment

| | Initial | | |
|---|---|---|---|
| Species Name | Value | Units | Description |
| V | 50.25 | picomole/liter | VEGF in retina |
| R | 0 | picomole/liter | drug in retina |
| VR | 0 | picomole/liter | Complex of VEGF-drug in retina |
| RVR | 0 | picomole/liter | Complex of drug-VEGF-drug in retina |
| deg_V | 0 | picomole/liter | VEGF eliminated from retina |
| deg_R | 0 | picomole/liter | drug eliminated from retina |
| deg_VR | 0 | picomole/liter | Complex of VEGF-drug eliminated from |
| | | | retina |
| deg_RVR | 0 | picomole/liter | Complex of drug-VEGF-drug eliminated |
| | | | from retina |
| FractionFreeVEGF | 1 | dimensionless | Used for analysis. Fraction of free |
| | | | VEGF in this compartment |

TABLE 3
Table of Species (state variables) in the Vitreous (Vit) Compartment.

| | Initial | | |
|---|---|---|---|
| Species Name | Value | Units | Description |
| V | 10.7318 | picomole/liter | VEGF in vitreous |
| R | 0 | picomole/liter | drug in vitreous |
| VR | 0 | picomole/liter | Complex of |
| | | | VEGF-drug |
| | | | in vitreous |
| RVR | 0 | picomole/liter | Complex of |
| | | | drug-VEGF-drug |
| | | | in vitreous |
| FractionFreeVEGF | 1 | dimensionless | Used for analysis. |
| | | | Fraction of free |
| | | | VEGF in this |
| | | | compartment |

TABLE 4
Table of Species (state variables) in the Aqueous (Aq) Compartment.

| | Initial | | |
|---|---|---|---|
| Species Name | Value | Units | Description |
| V | 1.7471 | picomole/liter | VEGF in aqueous |
| R | 0 | picomole/liter | drug in aqueous |
| VR | 0 | picomole/liter | Complex of VEGF-drug in aqueous |
| RVR | 0 | picomole/liter | Complex of drug-VEGF-drug in aqueous |
| deg_V | 0 | picomole/liter | VEGF eliminated from aqueous |
| deg_R | 0 | picomole/liter | drug eliminated from aqueous |
| deg_VR | 0 | picomole/liter | Complex of VEGF-drug eliminated from |
| | | | aqueous |
| deg_RVR | 0 | picomole/liter | Complex of drug-VEGF-drug eliminated |
| | | | from aqueous |
| FractionFreeVEGF | 1 | dimensionless | Used for analysis. Fraction of free VEGF |
| | | | in this compartment |

TABLE 5
Table of Parameters in the QSP model.

| Parameter | | Initial | | |
|---|---|---|---|---|
| Name | Value | Value | Units | Notes |
| koff | 21.6 | 21.6 | 1/day | single binding site |
| | | | | off rate (VR --> V + R) |
| Kd | 2070.5 | 2070.5 | picomole/liter | single binding site |
| | | | | model dissociation |
| | | | | constant |
| kon | 0 | 0.010432 | 1/((picomole/liter) * day) | single binding site |
| | | | | on rate (V + R --> |
| | | | | VR), set by rule |
| kon2 | 0 | 0.020865 | 1/((picomole/liter) * day) | 2 * kon correction for |
| | | | | antibody bivalency |
| koff2 | 0 | 43.2 | 1/day | 2 * koff correction for |
| | | | | antibody bivalency |
| vin | 17.5 | 17.5 | femtomole/day | rate of VEGF |
| | | | | production |
| cl | 3.6 | 3.6 | milliliter/day | clearance from the |
| | | | | aqueous chamber |
| sret | 9.71 | 9.71 | centimeter?2 | retinal surface area |
| thalf_V | 4.6366 | 4.6366 | day | ocular half-life of |
| | | | | VEGF |
| thalf_R | 0 | 4.4 | day | ocular half-life of |
| | | | | drug (set by rule) |
| thalf_VR | 0 | 6.1304 | day | ocular half-life of VR |
| | | | | complex (set by rule) |
| thalf_RVR | 0 | 7.1004 | day | ocular half-life of |
| | | | | RVR complex (set |
| | | | | by rule) |
| lambda_V | 0 | 0.14949 | 1/day | The long-time decay |
| | | | | rate of VEGF (set by |
| | | | | rule) |
| lambda_R | 0 | 0.15753 | 1/day | The long-time decay |
| | | | | rate of drug (set by |
| | | | | rule) |
| lambda_VR | 0 | 0.11307 | 1/day | The long-time decay |
| | | | | rate of VR complex |
| | | | | (set by rule) |
| lambda_RVR | 0 | 0.097621 | 1/day | The long-time decay |
| | | | | rate of RVR complex |
| | | | | (set by rule) |
| kel_V | 0 | 0.13024 | 1/day | elimination rate of |
| | | | | VEGF (set by rule) |
| kel_R | 0 | 0.138 | 1/day | elimination rate of |
| | | | | drug (set by rule) |
| kel_VR | 0 | 0.095237 | 1/day | elimination rate of |
| | | | | VR complex (set by |
| | | | | rule) |
| kel_RVR | 0 | 0.080516 | 1/day | elimination rate of |
| | | | | RVR complex (set |
| | | | | by rule) |
| rh_V | 2.39 | 2.39 | dimensionless | hydrodynamic radius |
| | | | | of VEGF (real units = |
| | | | | nanometer) |
| rh_R | 2.268 | 2.268 | dimensionless | hydrodynamic radius |
| | | | | of drug (real units = |
| | | | | nanometer) |
| rh_VR | 3.16 | 3.16 | dimensionless | hydrodynamic radius |
| | | | | of VR complex (real |
| | | | | units = nanometer) |
| rh_RVR | 3.66 | 3.66 | dimensionless | hydrodynamic radius |
| | | | | of RVR complex |
| | | | | (real units = |
| | | | | nanometer) |
| sec_in_day | 86400 | 86400 | second/day | Used in rule to |
| | | | | calculate |
| | | | | permeabilities |
| | | | | (cm/sec to cm/day) |
| prpe_exp | −0.48 | −0.48 | dimensionless | Used in rule to |
| | | | | calculate RPE |
| | | | | permeabilities |
| | | | | (exponent) |
| pilm_exp | −0.17 | −0.17 | dimensionless | Used in rule to |
| | | | | calculate ILM |
| | | | | permeabilities |
| | | | | (exponent) |
| prpe_factor | 4.04e−07 | 4.04e−07 | centimeter/second | Used in rule to |
| | | | | calculate RPE |
| | | | | permeabilities |
| | | | | (multiplier) |
| pilm_factor | 2.2e−07 | 2.2e−07 | centimeter/second | Used in rule to |
| | | | | calculate ILM |
| | | | | permeabilities |
| | | | | (multiplier) |
| prpe_V | 0 | 0.022975 | centimeter/day | RPE permeability of |
| | | | | VEGF (set by rule) |
| prpe_R | 0 | 0.02356 | centimeter/day | RPE permeability of |
| | | | | drug (set by rule) |
| prpe_VR | 0 | 0.020093 | centimeter/day | RPE permeability of |
| | | | | VR complex (set by |
| | | | | rule) |
| prpe_RVR | 0 | 0.018725 | centimeter/day | RPE permeability of |
| | | | | RVR (set by rule) |
| pilm_V | 0 | 0.016391 | centimeter/day | ILM permeability of |
| | | | | VEGF (set by rule) |
| pilm_R | 0 | 0.016538 | centimeter/day | ILM permeability of |
| | | | | drug (set by rule) |
| pilm_VR | 0 | 0.015631 | centimeter/day | ILM permeability of |
| | | | | VR complex (set by |
| | | | | rule) |
| pilm_RVR | 0 | 0.015246 | centimeter/day | ILM permeability of |
| | | | | RVR (set by rule) |
| sret_prpe_V | 0 | 0.22309 | milliliter/day | Rate of RPE |
| | | | | permeability of |
| | | | | VEGF (set by rule) |
| sret_prpe_R | 0 | 0.22877 | milliliter/day | Rate of RPE |
| | | | | permeability of drug |
| | | | | (set by rule) |
| sret_prpe_VR | 0 | 0.1951 | milliliter/day | Rate of RPE |
| | | | | permeability of VR |
| | | | | complex (set by rule) |
| sret_prpe_RVR | 0 | 0.18182 | milliliter/day | Rate of RPE |
| | | | | permeability of RVR |
| | | | | (set by rule) |
| sret_pilm_V | 0 | 0.15916 | milliliter/day | Rate of ILM |
| | | | | permeability of |
| | | | | VEGF (set by rule) |
| sret_pilm_R | 0 | 0.16058 | milliliter/day | Rate of ILM |
| | | | | permeability of drug |
| | | | | complex (set by rule) |
| sret_pilm_VR | 0 | 0.15178 | milliliter/day | Rate of ILM |
| | | | | permeability of VR |
| | | | | complex (set by rule) |
| sret_pilm_RVR | 0 | 0.14803 | milliliter/day | Rate of ILM |
| | | | | permeability of RVR |
| | | | | complex (set by rule) |
| EQ | 0 | 10.7318 | picomole/liter | common term used |
| | | | | in the calculation of |
| | | | | baseline steady- |
| | | | | state VEGF |
| EQ_unit | 1 | 1 | dimensionless | Used in the |
| | | | | calculation of the EQ |
| | | | | initial assignment |
| | | | | rule. Needed to |
| | | | | satisfy dimensional |
| | | | | analysis for the +1 |
| | | | | term |
| EQ_denom | 0 | 2.7824 | dimensionless | Used in the |
| | | | | calculation of the EQ |
| | | | | initial assignment |
| | | | | rule. This just moves |
| | | | | the long |
| | | | | denominator to its |
| | | | | own term |

TABLE 6
Table of Initial Assignment Rules in the QSP model′.

| | Initial | |
|---|---|---|
| Initial Assignments | Value | Notes |
| kon = koff/Kd | 0.010432 | VEGF + drug association rate |
| | | (monovalent) |
| kon2 = kon * 2 | 0.020865 | VEGF + drug association rate |
| | | (bivalent) |
| koff2 = koff * 2 | 43.2 | RVR dissociation rate (bivalent) |
| prpe_V = prpe_factor * sec_in_day * | 0.022975 | power law expression to calculate |
| rh_V^{∧}prpe_exp | | permeability from hydrodynamic |
| | | radius. (1) |
| prpe_R = prpe_factor * sec_in_day * | 0.02356 | power law expression to calculate |
| rh_R^{∧}prpe_exp | | permeability from hydrodynamic |
| | | radius. (1) |
| prpe_VR = prpe_factor * | 0.020093 | power law expression to calculate |
| sec_in_day * rh_VR^{∧}prpe_exp | | permeability from hydrodynamic |
| | | radius. (1) |
| prpe_RVR = prpe_factor * | 0.018725 | power law expression to calculate |
| sec_in_day * rh_RVR^{∧}prpe_exp | | permeability from hydrodynamic |
| | | radius. (1) |
| pilm_V = pilm_factor * sec_in_day | 0.016391 | power law expression to calculate |
| * rh_V^{∧}pilm_exp | | permeability from hydrodynamic |
| | | radius. (1) |
| pilm_R = pilm_factor * sec_in_day | 0.016538 | power law expression to calculate |
| * rh_R^{∧}pilm_exp | | permeability from hydrodynamic |
| | | radius. (1) |
| pilm_VR = pilm_factor * | 0.015631 | power law expression to calculate |
| sec_in_day * rh_VR^{∧}pilm_exp | | permeability from hydrodynamic |
| | | radius. (1) |
| pilm_RVR = pilm_factor * | 0.015246 | power law expression to calculate |
| sec_in_day * rh_RVR^{∧}pilm_exp | | permeability from hydrodynamic |
| | | radius. (1) |
| sret_prpe_V = sret * prpe_V | 0.22309 | Convert the permeability in cm/day |
| | | to mL/day by multiplying by the |
| | | retinal surface area. (cm^{∧}3 = mL) |
| sret_prpe_R = sret * prpe_R | 0.22877 | Convert the permeability in cm/day |
| | | to mL/day by multiplying by the |
| | | retinal surface area. (cm^{∧}3 = mL) |
| sret_prpe_VR = sret * prpe_VR | 0.1951 | Convert the permeability in cm/day |
| | | to mL/day by multiplying by the |
| | | retinal surface area. (cm^{∧}3 = mL) |
| sret_prpe_RVR = sret * prpe_RVR | 0.18182 | Convert the permeability in cm/day |
| | | to mL/day by multiplying by the |
| | | retinal surface area. (cm^{∧}3 = mL) |
| sret_pilm_V = sret * pilm_V | 0.15916 | Convert the permeability in cm/day |
| | | to mL/day by multiplying by the |
| | | retinal surface area. (cm^{∧}3 = mL) |
| sret_pilm_R = sret * pilm_R | 0.16058 | Convert the permeability in cm/day |
| | | to mL/day by multiplying by the |
| | | retinal surface area. (cm^{∧}3 = mL) |
| sret_pilm_VR = sret * pilm_VR | 0.15178 | Convert the permeability in cm/day |
| | | to mL/day by multiplying by the |
| | | retinal surface area. (cm^{∧}3 = mL) |
| sret_pilm_RVR = sret * pilm_RVR | 0.14803 | Convert the permeability in cm/day |
| | | to mL/day by multiplying by the |
| | | retinal surface area. (cm^{∧}3 = mL) |
| thalf_R = (thalf_V/rh_V) * rh_R | 4.4 | Half-life. Scaling from the VEGF |
| | | half and hydrodynamic radius (1) |
| thalf_VR = (thalf_V/rh_V) * rh_VR | 6.1304 | Half-life. Scaling from the VEGF |
| | | half and hydrodynamic radius (1) |
| thalf_RVR = | 7.1004 | Half-life. Scaling from the VEGF |
| (thalf_V/rh_V) * rh_RVR | | half and hydrodynamic radius (1) |
| lambda_V = | 0.14949 | Long-time decay rate (1) |
| 0.69314718056/thalf_V | | |
| lambda_R = | 0.15753 | Long-time decay rate (1) |
| 0.69314718056/thalf_R | | |
| lambda_VR = | 0.11307 | Long-time decay rate (1) |
| 0.69314718056/thalf_VR | | |
| lambda_RVR = | 0.097621 | Long-time decay rate (1) |
| 0.69314718056/thalf_RVR | | |
| kel_V = lambda_V − ((sret/Vit) * | 0.13024 | Ocular half-life to elimination rate (1) |
| pilm_V) + (((sret * pilm_V)^{∧}2)/ | | |
| (Vit * sret * (pilm_V + prpe_V) − | | |
| Vit * Ret * lambda_V)) | | |
| kel_R = lambda_R − ((sret/Vit) * | 0.138 | Ocular half-life to elimination rate (1) |
| pilm_R) + (((sret * pilm_R)^{∧}2)/ | | |
| (Vit * sret * (pilm_R + prpe_R) − | | |
| Vit * Ret * lambda_R)) | | |
| kel_VR = lambda_VR − ((sret/Vit) * | 0.095237 | Ocular half-life to elimination rate (1) |
| pilm_VR) + (((sret * | | |
| pilm_VR)^{∧}2)/ | | |
| (Vit * sret * (pilm_VR + prpe_VR) − | | |
| Vit * Ret * lambda_VR)) | | |
| kel_RVR = lambda_RVR − | 0.080516 | Ocular half-life to elimination rate (1) |
| ((sret/Vit) * pilm_RVR) + (((sret * | | |
| pilm_RVR)^{∧}2)/ | | |
| (Vit * sret * (pilm_RVR + prpe_RVR) − | | |
| Vit * Ret * lambda_RVR)) | | |
| EQ_denom = | 2.7824 | common term used in the calculation |
| (sret * prpe_V)/(Vit * kel_V) + | | of baseline steady-state VEGF |
| EQ_unit + (prpe_V/pilm_V) | | |
| EQ = 1/(Vit * kel_V) * (vin/ | 10.7318 | common term used in the calculation |
| EQ_denom ) | | of baseline steady-state VEGF |
| Ret. V = EQ * (EQ_unit + | 50.25 | baseline steady-state VEGF in retina |
| kel_V/((sret/Vit) * pilm_V)) | | |
| Vit.V = EQ | 10.7318 | baseline steady-state VEGF in retina |
| Aq.V = EQ * Vit/cl * kel_V | 1.7471 | baseline steady-state VEGF in retina |

TABLE 7
Table of Repeated Assignment Rules in the QSP model.

| | Initial | |
|---|---|---|
| Repeated Assignments | Value | Notes |
| Vit.FractionFreeVEGF = Vit.V/ | 1 | Calculate the fraction of |
| (Vit.V + Vit.VR + Vit.RVR) | | VEGF that is free in vitreous |
| Ret.FractionFree VEGF = Ret.V/ | 1 | Calculate the fraction of |
| (Ret.V + Ret.VR + Ret.RVR) | | VEGF that is free in retina |
| Aq.FractionFree VEGF = Aq.V/ | 1 | Calculate the fraction of |
| (Aq.V + Aq.VR + Aq.RVR) | | VEGF that is free in aqueous |

### Model Parameters

Amongst all parameters employed in the model (Tables 5 to 7), some pertain to the drug that is being modeled, i.e., brolucizumab. These are the brolucizumab: VEGF binding parameter values (the dissociation rate constant “koff”, the association rate constant “kon” and the equilibrium binding constant “Kd”) and the hydrodynamic radius values for drug (“rh_R”) and associated drug: VEGF complexes (“rh_VR” and “rh_RVR”).

The brolucizumab: VEGF binding parameter values (Table 8) were defined on the basis of a surface plasmon resonance (SPR) in vitro binding experiment with drug and recombinant VEGF165 protein at 37° C. These in vitro measured brolucizumab: VEGF binding parameter values were then scaled similar to ranibizumab to obtain in vivo estimates. A scaling factor of 20.5 is employed based up on the in vitro measured Kd for ranibizumab and the model fit (in vivo) Kd given by Hutton-Smith et al., 2018 using the hydrodynamic radius of 3.0 nm and ocular half-life of 5.8 days for ranibizumab given by Caruso et al., 2020. The scaling is performed such that the koff is kept constant and the kon is divided by the scaling factor.

TABLE 8
Binding kinetics of brolucizumab and ranibizumab to human VEGF165 at 37º C.

| | | | | Kd, in vivo (pM), |
|---|---|---|---|---|
| VEGF | | | Kd, in | with 20.5× scaling |
| inhibitor | kon (1/Ms) | koff (1/s) | vitro (pM) | factor |
| brolucizumab | (2.5 + 0.1) × 10⁶ | (2.5 + 0.1) × 10⁻⁴ | 101 + 2 | 2070 |
| ranibizumab | (5.8 + 0.1) × 10⁴ | <1 × 10⁻⁵ | <171 + 2 | 3500 |

The hydrodynamic radius values for drug (“rh_R”) and for VEGF (“rh_V”) and the associated ocular half-lives for drug (“thalf_R”) and for VEGF (“thalf_V”) are established in accordance with the correlations provided by Caruso et al., 2020 through an extensive meta-analysis of published data. These correlations provide a relationship between hydrodynamic radius and ocular halflife and used here to calculate either of these parameters where the other is known. Particularly we rely on the ocular half-life value of 4.4 days for the drug provided by the clinical pharmacology analysis and the hydrodynamic radius for VEGF of 2.39 nm estimated by Hutton-Smith et al., 2018. The hydrodynamic radius values for associated drug: VEGF complexes (“rh_VR” and “rh_RVR”) were sourced from computational structure models of drug and VEGF. All hydrodynamic radius and half-life values are presented in Table 9.

TABLE 9
Hydrodynamic radius and half-life for all species.

| Species | Rh (nm) | Thalf (days) |
|---|---|---|
| V | 2.39 | 4.64 |
| R | 2.27 | 4.40 |
| VR | 3.16 | 6.13 |
| RVR | 3.66 | 7.10 |

Drug-independent parameters used are used to describe VEGF production and distribution within and out of the eye, as well as general biophysical properties of the eye such as permeability coefficients for the Inner Limiting Membrane (ILM) and Retinal Pigment Epithelium (RPE) and the clearance rate from the aqueous chamber. These values are shown in Table 5. Simulations are performed with varying retinal VEGF synthesis rates to consider intra-patient variability in ocular VEGF levels (Table 10). To represent the low, mean and high retinal VEGF synthesis rates, the conditions of mean +/−2× SD of the population distribution reported in Hutton-Smith et al., 2018 are employed.

TABLE 10
Retinal VEGF synthesis rates and baseline levels.

| | | | Baseline VEGF |
|---|---|---|---|
| VEGF | | VEGF synthesis | concentration |
| Synthesis | Calculation | rate (fmol/day) | (pM) |
| Low | Mean − 2 × standard | 3.7 | 10.6 |
| | deviation | | |
| Mean | Mean | 17.5 | 50.3 |
| High | Mean + 2 × standard | 31.3 | 89.9 |
| | deviation | | |

### Modeling Steps and Process Overview

Simulations were performed with drug administration regimens as indicated in each of the figures. The PK/PD profiles of the concentration of drug in the vitreous over time (PK) and of free VEGF in the retina (PD) were simulated and results plotted as graphs. Simulations are performed with varying retinal VEGF synthesis rates to consider inter-patient variability in ocular VEGF levels. Intraocular PK/PD and retinal VEGF inhibition is first simulated for the clinical dosing regimens defined by the HAWK and HARRIER studies (3× Q4W loading phase followed by Q8W or Q12W maintenance phase). As a second step, retinal VEGF inhibition is simulated for the alternative dosing regimen of 2× Q6W loading phase followed by Q8W or Q12W maintenance phase.

### Results

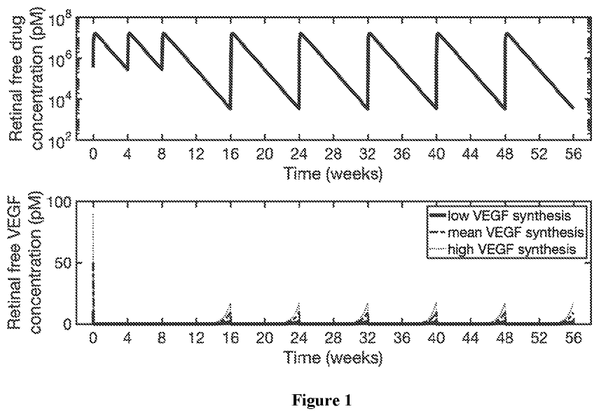

Simulated retinal free drug concentrations and the retinal free VEGF concentrations for the dosing regimen of 3× Q4W loading phase followed by Q8W maintenance phase is represented in FIG. 1.

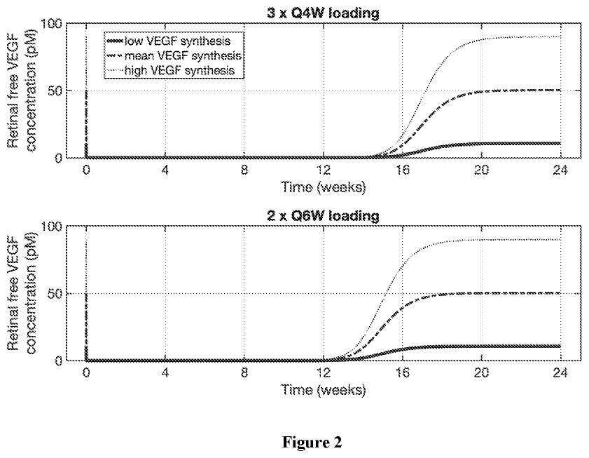

Simulated retinal VEGF inhibition for loading dose regimens of 3× Q4W and 2× Q6W are represented in FIG. 2. The results demonstrate that both regimens provide almost complete VEGF inhibition (near zero retinal VEGF concentrations) over the first 12 to 13 weeks. Recovery of free VEGF is observed to begin approximately 6 to 7 weeks following the last dose of each regimen and is consistent with clearance of the drug from the eye.

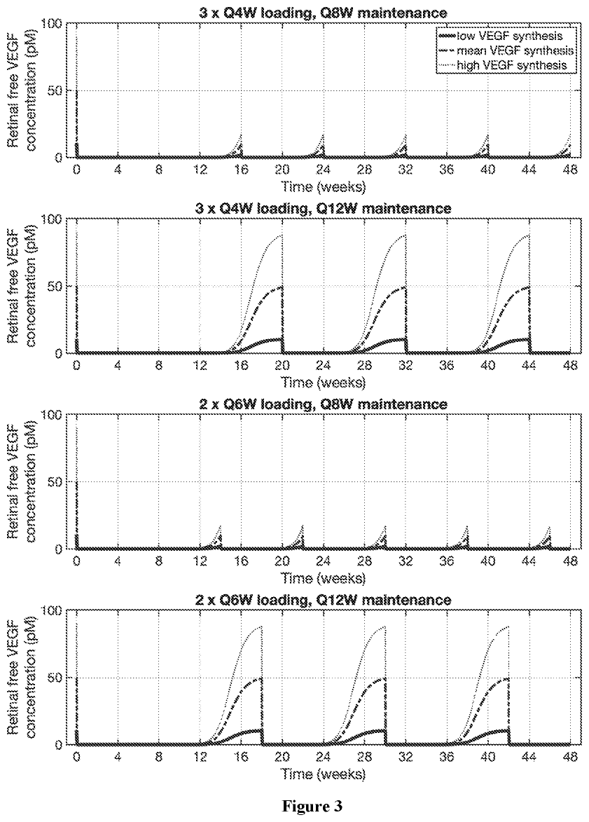

The retinal VEGF inhibition for both loading dose regimens (3× Q4W and 2× Q6W) followed by either Q8W or Q12W maintenance doses was simulated and represented in FIG. 3. It was observed that there is no apparent difference in VEGF inhibition in the maintenance period following either loading dose regimen (3× Q4W or 2× Q6W).

Several observations are apparent in the results. First, a 2× Q6W loading regimen is predicted to elicit substantially similar (near-complete) retinal VEGF inhibition as 3× Q4W regardless of the baseline VEGF levels. Second, there is no apparent difference in drug PK or VEGF inhibition in the maintenance period following either loading dose regimen (3× Q4W or 2× Q6W). Third, the extent of free retinal VEGF recovery with Q8W and Q12W maintenance dosing is more apparent under conditions of high VEGF synthesis.

### Conclusions

It is observed that, for the loading phase, 3× Q4W and 2× Q6W provide almost complete VEGF inhibition (near zero retinal VEGF concentrations), with recovery of free VEGF beginning approximately 6 to 7 weeks after the last dose. A complete recovery to baseline levels of VEGF is predicted to occur at approximately 12 weeks after the last dose. The pharmacodynamic response during the maintenance phase is predicted to be identical for both loading dose regimens. A maintenance regimen of Q8W dosing leads to partial recovery of retinal VEGF before administration of consecutive doses, whereas Q12W dosing allows for almost full recovery of VEGF levels to pre-administration steady state levels before administration of consecutive doses. These observations are conserved for the conditions of low, median, and high levels of VEGF, which is expected given the high excess of brolucizumab over VEGF (approximately >100,000 fold).

Together, these posology simulations suggest that reduced dose intensity within the loading period may still maintain robust VEGF inhibition and that patient factors such as intraocular VEGF levels may influence the ability to maintain VEGF inhibition with prolonged dosing intervals in the maintenance period. The simulations further suggest that a switch to a two-dose loading regimen (2× Q6W followed by maintenance doses) may achieve similar VEGF inhibition as the approved three-dose loading regimen (3× Q4W followed by maintenance doses).

### Example 2: Clinical Trial Simulations of q6w Loading and Individualized q12w/q8w Brolucizumab Treatment Effect on Retinal Thickness and Visual Acuity in Wet AMD Patients

### Objectives

The aim was to simulate reduced loading with brolucizumab 6 mg dosing with two or three injections 6 weeks apart followed by individualized q12w/q8w maintenance for the patient population of the HAWK and HARRIER trials and compare BCVA gain and CSFT reduction to the results of brolucizumab 6 mg treatment arms of the HAWK and HARRIER trials with 3 monthly loading doses followed by individualized q12w/q8w maintenance.

### Data

Clinical trial simulations of two or three q6w loading doses and three q4w loading doses followed by q12w/q8w maintenance in patient population of HAWK and HARRIER studies.

The HAWK study was a two-year, randomized, double-masked, three arm Phase III registration study comparing the efficacy and safety of brolucizumab (3 mg and 6 mg) versus aflibercept 2 mg in 1078 patients with nAMD.

The HARRIER study was a two-year, randomized, double-masked, two-arm Phase III registration study comparing the efficacy and safety of 6 mg brolucizumab versus aflibercept 2 mg in 739 patients with nAMD.

In the HAWK and HARRIER studies, patients in brolucizumab arms, after 3 monthly loading doses, were initially assigned to q12w regimen and switched to q8w regimen according to individual treatment need after disease activity assessment (DAA) at predefined visits. The DAA visits in the first year of HAWK study were Weeks 16, 20, 32, 44; the HARRIER study had additional DAA at Weeks 28 and 40.

### Methods

Non-linear mixed effects PK/PD model was developed and used to simulate the longitudinal dynamics of CSFT and BCVA change from baseline in wet AMD patients treated with anti-VEGF. The PK of anti-VEGF drugs was described by one-compartmental models where brolucizumab vitreal elimination half-life was fixed to typical value of 8.6 days obtained from a population PK analysis, while typical aflibercept half-life of 5.9 days was estimated using K-PD approach. Central subfield foveal thickness (CSFT) was modelled as a sum of time-independent normal thickness Rₙₒᵣₘ and disease induced thickness R(t) that can be reduced by anti-VEGF treatment. The effect of anti-VEGF on R(t) was described using generalized growth model (1), while BCVA improvement was governed by CSFT reduction after delay using an effect compartment. The model included IIV on normal CSFT Rnorm, baseline disease related CSFT Rdis, drug effect on CSFT Emax, CSFT growth rate k, CSFT reduction effect on BCVA Vmax and CSFT reduction effect delay on BCVA k_{eff}. Covariate effects of baseline CSFT on Rdis and baseline BCVA on Vmax and individual residuals were included into the model. The drug effect parameter Emax was modelled as drug specific, while EC50 was modelled as common to all three drugs. The full joint model of CSFT(t) and BCVA change from baseline V(t) is specified by the set of following equations.

$$For PK : \qquad ( 1 )$$

$$\frac{dC ( t )}{dt} = {k}_{elim} C ( t ) ,$$

$$C ( 0 ) = 0$$

$$For CSFT :$$

$$\frac{dr ( t )}{dt} = k \cdot {r}^{β} ( t ) \cdot ( 1 - E max \frac{C ( t )}{{EC}_{50} + C ( t )} - r ( t ) ) ,$$

$$β > 0 ,$$

$$r ( 0 ) = 1$$

$$CSFT ( t ) = {R}_{norm} + Rdis \cdot r ( t )$$

For BCVA change from baseline V(t) with delayed effect:

$$\frac{{dr}_{eff} ( t )}{dt} = {k}_{eff} \cdot ( 1 - r ( t ) - {r}_{eff} ( t ) ) , \qquad ( 2 )$$

$${r}_{eff} ( t = 0 ) = 0$$

$$V ( t ) = V max \cdot {r}_{eff}^{γ} ( t )$$

Inter-individual variability of model parameters and covariate effects are of the form:

$${R}_{norm , i} = {R}_{norm} \cdot {e}^{{η}_{1 , i}}$$

$${R}_{dis , i} = {R}_{dis} \cdot {max ( \frac{BRT - 240}{200} , 1 )}^{{θ}_{1}} \cdot {e}^{{η}_{2 , i}}$$

$${k}_{i} = k \cdot {e}^{{η}_{3 , i}}$$

$$E {max}_{i} = E max \cdot {e}^{{η}_{4 , i}}$$

$$V {max}_{i} = V max + {θ}_{2} ( BVA - 60 ) + {θ}_{3} ( AGE - 77 ) + {η}_{5 , i}$$

$${k}_{eff , i} = {k}_{eff} \cdot {e}^{{η}_{6 , i}}$$

where BRT is baseline CSFT in um, BVA is baseline BCVA in letters. The residual error models are:

$${CSFT}_{ij} = ( {R}_{norm , i} + {R}_{dis , ij} ) ( 1 + {σ}_{1} ) {ε}_{ij}$$

$${dBCVA}_{ij} = {V}_{ij} + {σ}_{2 , i} \cdot {ε}_{ij}$$

$${σ}_{2 , i} = {σ}_{2} \cdot exp ( {θ}_{4} ( BVA - 60 ) + {η}_{7 , i} )$$

where η—independent normally distributed random effects, εᵢⱼ are normally distributed random variables with mean 0 and standard deviation 1, σ₁ is standard deviation of CSFT residuals, and σ_{2,i} is individual standard deviations of BCVA residuals.

### Model Evaluation for One Year of q8w Treatment

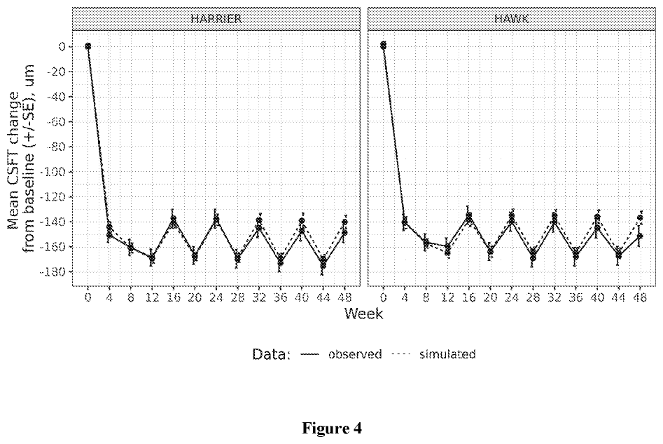

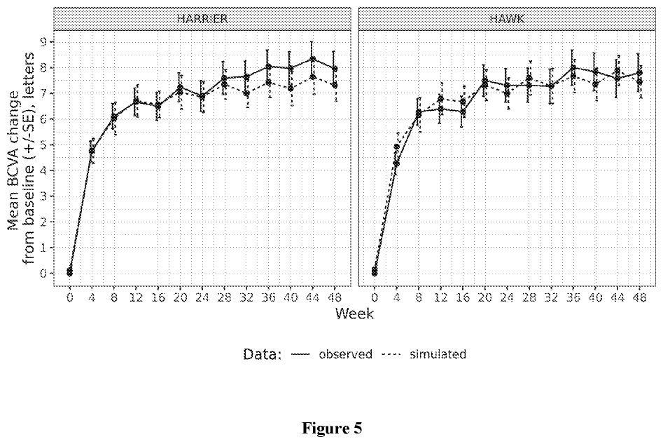

The PK/PD model was developed using HAWK and HARRIER data up to first treatment individualization visit at Week 16 in brolucizumab treatment arms. The aflibercept treatment arms data was also fitted by model up to Week 16 to avoid imbalances. One year of aflibercept 2 mg treatment arms of HAWK and HARRIER, i.e., using patient covariates of the aflibercept arms and 3 q4w loading followed by q8w maintenance, was simulated. The results are presented in FIG. 4 and FIG. 5. It was observed that CSFT change from baseline replicates the observed data well. BCVA change from baseline is slightly underestimated by the model for the HARRIER study, however the mean of the observed data was within SE of the simulated mean BCVA change from baseline.

### Disease Activity Assessment Simulations

Brolucizumab treatment in the HAWK and HARRIER studies was individualized for q12w or q8w intervals based on disease activity (DA) assessment at Week 16 and at the end of 12 week intervals in HAWK (Week 16, 20, 32 and 44 visits in the first year). In the HARRIER study DA assessments were additionally performed at the end of 8 week intervals (Week 16, 20, 28, 32, 40 and 44 visits in the first year). The disease activity assessment criteria were not defined, however the guidelines to reducing treatment interval from q12w to q8w were provided. At Week 16 they were:
Decrease in BCVA of ≥5 letters compared with Baseline
Decrease in BCVA of ≥3 letters and CSFT increase ≥75 μm compared with Week 12
Decrease in BCVA of ≥5 letters due to nAMD DA compared with Week 12
New or worse IRF/intraretinal cysts compared with Week 12

After Week 16 the guidance was:
Decrease in BCVA of ≥5 letters due to nAMD DA compared with Week 12

These guidelines allow for investigators discretion in treatment decisions. Current standard of anti-VEGF treatment individualization is largely guided by OCT (Fung, A. E., et al., 2007. American journal of ophthalmology, 143(4), pp.566-583). Thus, DA assessments and treatment interval individualization was approximated using the simulated dynamics of CSFT, which is the best available proxy to quantify retinal fluid in OCT images.

In the single ascending brolucizumab dose SEE study, switch for standard of care was recommended if CSFT exceeded 340 μm threshold. In phase IIIb SUSTAIN study of ranibizumab administered under pro-re-nata (PRN) regimen, the retreatment criteria were BCVA drop of more than 5 letters or retinal thickness increase over 100 μm compared to best values achieved in first 4 months (Holz, F. G., et al., 2011. Ophthalmology, 118(4), pp.663-671). The SUSTAIN criteria were likely too relaxed as mean BCVA improvement from baseline to month 12 was 3.6 letters, which is below typical visual acuity improvement of 5-6letters in anti-VEGF clinical trials with similar mean baseline BCVA values (Khanna, S., et al., 2019. BMJ open ophthalmology, 4(1), p.e000398). The HAWK and HARRIER studies used threshold of 75 um CSFT increase from Week 12, which is maximum treatment effect visit, in guidance for DA assessments. Thresholds of 50 um and 75 um increase from mean and minimal of CSFT at two previous visits respectively were recommended for DA assessment in recent studies in neovascular AMD (Heier, J. S., et al., 2022. The Lancet).

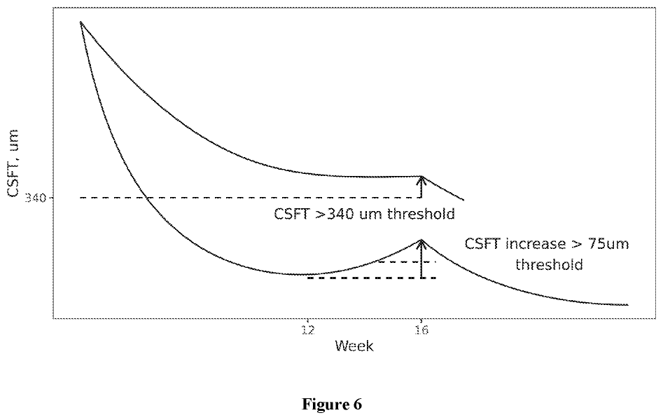

Various treatment scenarios were simulated: when patients were switched to q8w based on DA presence, where DA presence was simulated as either CSFT exceeding a specific CSFT threshold or CSFT increase from Week 12 (maximum loading effect visit) is above a CSFT increase threshold. FIG. 6 is an illustration of simulated disease activity presence. The CSFT value threshold is aimed to simulate DA presence in slow responders, while CSFT increase threshold is aimed to detect recurrence of active disease.

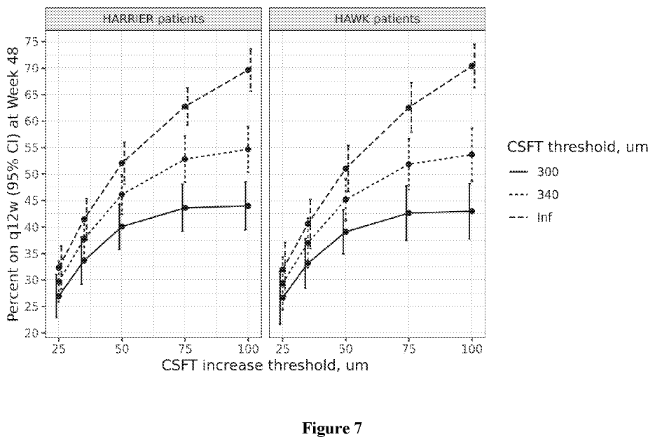

Percent of patients was estimated that remain on q12w treatment after 3 q4w loading doses in the HAWK and HARRIER brolucizumab 6 mg arms patient population. Each study was simulated 20 times to estimate standard errors. The drop out of patients in 6 mg brolucizumab arms of HAWK and HARRIER studies was about 9% and was not simulated. The DA assessment visits where DA assessment and regimen switch were simulated correspond to DA assessment visits in the HAWK study (Week 16, 20, 32 and 44 visits). FIG. 7 shows percent of patients still on q12w regimen at Week 48 for various CSFT and CSFT increase thresholds. In the HAWK trial the actual percentage (95% CI) was 55.6 (50.2,60.8), while in the HARRIER study it was 51.0 (45.7, 56.1).

From FIG. 7 it follows that CSFT threshold of 340 μm and CSFT increase threshold of 75 μm reproduces percentages of patients on q12w in HAWK and HARRIER within confidence intervals. These thresholds had been used in clinical trials and thus are clinically meaningful.

For applicability of these thresholds to simulations of various loading regimen, they must also replicate the efficacy results for CSFT and BCVA for brolucizumab 6 mg arms of the HAWK and HARRIER trials.

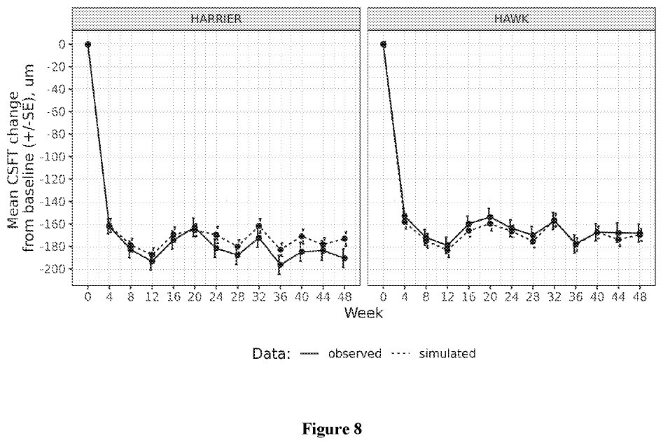

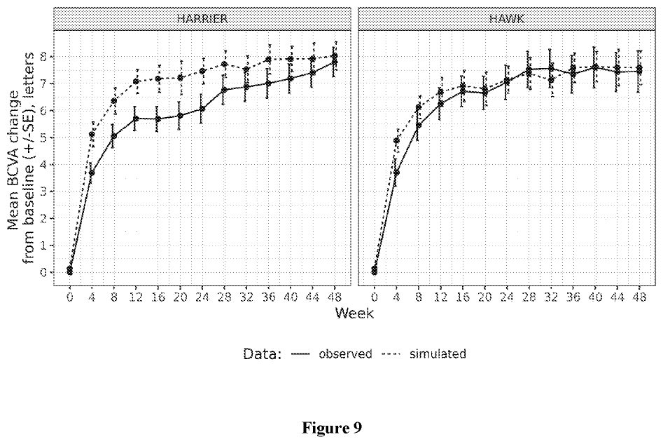

Simulations of mean CSFT and BCVA change from baseline at 3 q4w loading brolucizumab 6 mg doses followed by individualized q12w/q8w treatment are presented in FIG. 8 and FIG. 9. While there is good agreement between simulated and observed CSFT and BCVA in the HAWK study, efficacy results in simulated HARRIER study show some deviation from observed data: simulated mean CSFT improvement is slightly less than the mean of observed data and simulated mean BCVA improvement appears better than the mean observed data for the first treatment months. Simulated and observed mean BCVA improvements are within their confidence intervals only towards the end of the first year of treatment. Since aflibercept arms in both studies and brolucizumab 6 mg simulations in HAWK study are in good agreement with the observed data for both efficacy metrics, these simulations were considered to be an adequate representation of the treatment effect in the treated population.

Thus, positive DA assessment followed by switch to q8w treatment when simulated as either increase of CSFT above 340 μm or CSFT increase from Week 12 above 75 μm well replicates HAWK and HARRIER efficacy data in terms of CSFT reduction, BCVA improvement and percent of patients on q12w at Week 48.

### Results

### Simulation of Two or Three q6w Loading Doses and q12w/q8w Maintenance

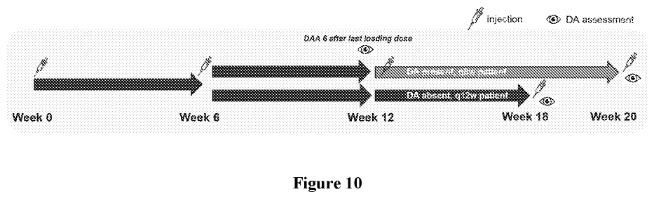

Two q6w loading doses of brolucizumab 6 mg followed by individualized q12w/q8w maintenance were simulated in populations of brolucizumab 6 mg treatment arm of the HAWK and HARRIER studies. At Week 12, six weeks after second loading injection, DA assessment was simulated. If DA was present a patient was given an injection and patients was treated q8w thereafter. If DA was absent, patient was scheduled for a visit at Week 18, 12 weeks from last loading dose, and DA was again assessed. At this visit patient was treated independently of DA, but if DA was present then patient was switched to q8w, otherwise patient was treated q12w with DA assessment at the end of each 12 week interval. At any subsequent positive DA assessments patients were switched to q8w treatment. DA presence was simulated as either CSFT exceeding 340 μm or CSFT increase from Week 12 is above a 75 μm. Schematics of simulated DA assessments and treatment schedule is presented in FIG. 10.

Each study was simulated 20 times to estimate standard errors of the means of simulated values—BCVA change from baseline, CSFT change from baseline and percent of patients on q12w treatment at Week 48. The drop out of patients in 6 mg brolucizumab arms of HAWK and HARRIER studies was about 9% and it was not simulated.

The simulated CSFT change from baseline and BCVA change from baseline after two q6w loading injections and individualized q12w/q8w treatment were close to the HAWK and HARRIER study data, except for slower than expected BCVA improvement observed in brolucizumab 6 mg treatment arm of HARRIER as described earlier. We compared simulated BCVA and CSFT changes from baseline at Week 48, which was primary endpoint visit for those studies, and mean BCVA and CSFT changes from baseline between Week 36 and Week 48 with the values from the HAWK and HARRIER clinical studies reports.

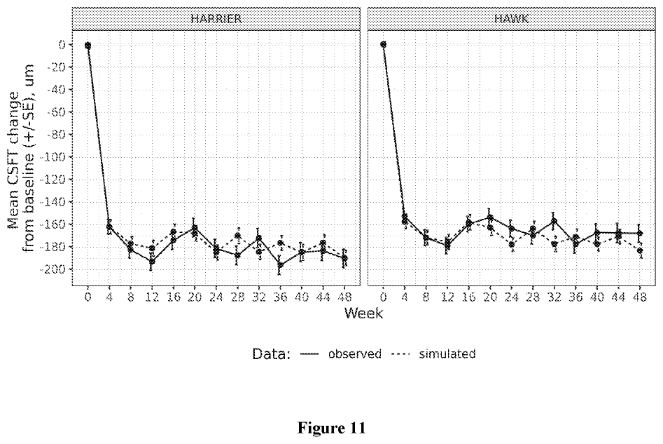

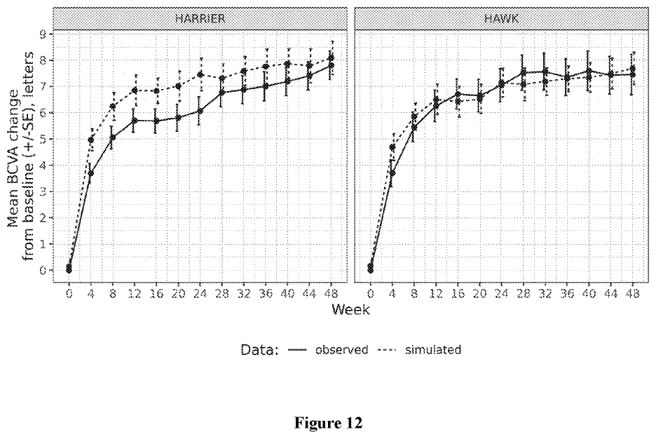

There was no statistically significant difference between simulated results and the observed data (FIG. 11 and FIG. 12, Table 11 and Table 12). The percentages of patients on q12w treatment interval at the end of simulated studies (Week 48) in brolucizumab 6 mg HAWK and HARRIER populations were 50% (2% SE), which is only slightly lower than 55.6% and 51.0% reported for the HAWK and HARRIER respectively.

TABLE 11
Comparison of simulated and observed mean CSFT change from baseline
at Week 48 and over the period from Week 36 to Week 48.

| | | Simulated CSFT | Observed CSFT | P-value¹ of |
|---|---|---|---|---|
| Study | Time/Interval | change (SE), um | change (SE), um | difference |
| HAWK | Week 48 | −183.0(6.96) | −170.9(7.51) | 0.08 |
| | Weeks 36-48 | −175.4(6.62) | −170.1(7.56) | 0.43 |
| HARRIER | Week 48 | −190.2(7.08) | −189.8(8.23) | 0.95 |
| | Weeks 36-48 | −182.6(7.29) | −187.4(8.09) | 0.51 |

Source:/vob/CRTH258A/mas/mas_2/model/pgm_001/ftva/sim_2q6load_q12q8.RCSR Tables 14.2-15.1, 14.2-18.1 for both HAWK and HARRIER (LOCF, FAS)
¹P-value for two-tailed test of difference of simulated and observed means of normal variable with standard deviation of simulated SE

TABLE 12
Comparison of simulated and observed mean BCVA change from
baseline at Week 48 and over the period from Week 36 to Week 48.

| | | Simulated | Observed | |
|---|---|---|---|---|
| | | BCVA | BCVA | |
| | | change | change | |
| | | (SE), | (SE), | P-value of |
| Study | Time/Interval | letters | letters | difference |
| HAWK | Week 48 | 7.7(0.61) | 7.2(0.79) | 0.42 |
| | Weeks 36-48 | 7.4(0.55) | 7.2(0.73) | 0.68 |
| HARRIER | Week 48 | 8.1(0.63) | 7.5(0.57) | 0.35 |
| | Weeks 36-48 | 7.9(0.60) | 6.9(0.57) | 0.1 |

Source:/vob/CRTH258A/mas/mas_2/model/pgm_001/ftva/sim_2q6load_q12q8.RCSR Tables 14.2-1.3, 14.2-2.3 for both HAWK and HARRIER (MMRM, FAS)
¹P-value for two-tailed test of difference of simulated and observed means of normal variable with standard deviation of simulated SE

### Discussion

FIG. 4 and FIG. 5 demonstrate that the joint PK/PD model for CSFT and BCVA can adequately simulate the data for one year of aflibercept treatment at q8w regimen. Further, after selecting clinically relevant CSFT thresholds of simulated DA assessments for treatment interval individualization, the model well reproduces CSFT and BCVA efficacy results of the individualized q8w/q12w treatment after 3 q4w loading doses of brolucizumab 6 mg treatment arms of the HAWK and HARRIER studies (FIG. 8, FIG. 9). Two q6w loading doses with optional third dose at Week 12 followed by q12w/q8w maintenance governed by same DA assessment rules as in simulations replicating brolucizumab 6 mg treatment arms of the HAWK and HARRIER trials were simulated. This led to similar efficacy results in terms of BCVA improvement and CSFT reduction as was observed in the trials. Same proportion or only about 5% less patients on q12w maintenance at Week 48 compared to the HARRIER and HAWK study respectively were computed. Percent (SE) of simulated patients required third loading dose at Week 12 was 20.4(1).

### Conclusion

Reducing brolucizumab 6 mg treatment during loading phase from 3 q4w doses to 2 or 3 q6w doses is expected to lead to similar efficacy at the cost of a <5% reduction of patients on q12w maintenance treatment.

1. A method for treating neovascular age-related macular degeneration (nAMD) in a patient, the method comprising:
(a) administering to the patient as a loading phase of two individual doses of brolucizumab at 6-week interval (q6w regimen), and
(b) assessing the patient for disease activity between ≥0 and ≤6 weeks after the second dose of the loading phase.

2. The method of claim 1, wherein if presence of disease activity is identified after the second dose of brolucizumab, the method further comprises administering to the patient as part of the loading phase a third dose of brolucizumab 6 weeks after the second dose.

3. The method of claim 1, comprising administering to the patient after the loading phase a maintenance phase of one or more additional individual doses of brolucizumab, wherein each additional dose is administered at an administration interval of at least once every 8 weeks (q8w regimen).

4. The method of claim 1, comprising administering to the patient after the loading phase a maintenance phase of one or more additional individual doses of brolucizumab, wherein each additional dose is administered at an administration interval of at least once every 12 weeks (q12w regimen).

5. The method of claim 3, comprising assessing the patient for disease activity during the maintenance phase and administering to the patient additional doses at an administration interval of once every 8 weeks (q8w regimen) when there is disease activity observed and administering to the patient additional doses at an administration interval of once every 12 weeks (q12w regimen) when there is no disease activity observed.

6. A method for treating neovascular age-related macular degeneration (nAMD) in a patient, the method comprising:
(a) administering to the patient two individual doses of brolucizumab at 6-week interval (q6w regimen); and
(b) administering to the patient one or more additional doses of brolucizumab, wherein each additional dose is administered in an administration interval of at least 8 weeks after the immediately preceding dose.

7. The method of claim 6, wherein the method further comprises:
assessing the patient for disease activity after the second dose of brolucizumab between ≥0 and ≤6 weeks after the second dose of brolucizumab.

8. The method of claim 7, wherein if presence of disease activity is identified after the second dose of brolucizumab, the method further comprises administering to the patient a third dose of brolucizumab 6 weeks after the administration of the second dose (q6w regimen).

9. The method of claim 6, wherein the method does not comprise administering to the patient more than 3 doses in an administration interval of less than 8 week.

10. The method of claim 7, wherein the method further comprises assessing the patient for disease activity before or after administering every q8w or q12w dose of brolucizumab.

11. The method of claim 10, wherein if presence of disease activity is identified after a q12w dose of brolucizumab, the patient is switched to a q8w regimen of brolucizumab.

12. The method of claim 7, wherein the disease activity is assessed based on one or more of the following:
(i) best corrected visual acuity (BCVA),
(ii) visual acuity (VA),
(iii) central subfield thickness (CSFT), and/or
(iv) presence of intraretinal cysts/fluid.

13. The method of claim 12, wherein the presence of disease activity includes one or more of the following:
(i) decrease in Best Corrected Visual Acuity (BCVA),
(ii) decrease in Visual Acuity (VA),
(iii) increase or lack of reduction in Central Subfield Thickness (CSFT),
(iv) new or persistent or recurrent Intraretinal Cysts (IRC) and/or Intraretinal Fluid (IRF) and/or Subretinal Fluid (SRF).

14-17. (canceled)

18. The method of claim 6, wherein brolucizumab is administered by an intravitreal injection.

19. The method of claim 18, wherein the dose of brolucizumab is from about 3 mg to about 6 mg.

20-29. (canceled)
